(19) United States
(12) Patent Application Publication
(10) Pub. No.: US 2026/0251654 A1
(43) Pub. Date: Aug. 27, 2026

(54) ASSAYS AND REAGENTS FOR THE DETECTION OF SOLUBLE GP120

(71) Applicants: VAL-CHUM, LIMITED PARTNERSHIP, Québec (CA); THE HENRY M. JACKSON FOUNDATION FOR THE ADVANCEMENT OF MILITARY MEDICINE, INC., Bethesda, MD (US)

(72) Inventors: Andrés FINZI, Saint-Bruno-de-Montarville (CA); Marzena PAZGIER, Derwood, MD (US); Jonathan RICHARD, Boucherville (CA); Mehdi BENLARBI, Gatineau (CA)

(21) Appl. No.: 19/162,114

(22) PCT Filed: Mar. 6, 2024
(86) PCT No.: PCT/CA2024/050272

§ 371 Date: Sep. 4, 2025

Related U.S. Application Data
(60) Provisional application No. 63/450,437, filed on Mar. 7, 2023.

(51) Int. Cl. G01N 33/569; A61K 31/496; A61K 31/675; C07K 16/114

(52) U.S. Cl. CPC G01N 33/56988; A61K 31/496; A61K 31/675; C07K 16/1145; G01N 2333/162; G01N 2469/10

(57) ABSTRACT

Soluble gp120 (sgp120) is associated to HIV-1-induced immune dysfunction, such as chronic immune activation. However, the detection of sgp120 in biological fluids such as plasma, serum, cervicovaginal fluids, and cerebrospinal fluids is challenging. The present application relates to novel methods and kits for the detection of sgp120 in biological fluids. These methods and kits are based on the use of specific combinations of sgp120-binding molecules recognizing particular domains of sgp120, notably the N-terminal 8-stranded β-sandwich structure, the constant region 1 and 2 (C1-C2) portion, the bridging sheet of the Co-Receptor Binding Site (CoRBS), and/or the CD4-binding domain.

### CROSS REFERENCE TO RELATED APPLICATIONS

The present application claims the benefit of U.S. provisional patent application Ser. No. 63/450,437 filed on Mar. 7, 2023, which is incorporated herein by reference in its entirety.

### STATEMENT OF GOVERNMENT SUPPORT

This invention was made with government support under Grant No. R01A1129769 and R01AI116274 awarded by the National Institutes of Health. The government has certain rights in the invention.

### SEQUENCE LISTING

A sequence listing is submitted herewith as an XML file named G17004-00018_Seq Listing.xml, created on Mar. 6, 2024, and having a size of ~49100 bytes. The content of the aforementioned file is hereby incorporated by reference in its entirety.

### TECHNICAL FIELD

The present invention generally relates to the field of viral infections and diseases, and more particularly to the detection of residual human immunodeficiency virus (HIV) infection and/or antigen.

### BACKGROUND

While antiretroviral therapy (ART) efficiently inhibits viral replication, people living with human immunodeficiency virus (HIV; PLWH) have a 15-year gap in comorbidity-free years [1]. The underlying causes for this gap are numerous and include the presence of residual immune dysfunction and chronic antigenic stimulation by HIV, which contributes to a state of sustained inflammation [2]. Persistent immune dysfunction has also been associated with the immunological nonresponse that many PLWH experience [3-7]. These individuals, despite efficient viral control, have failure to restore circulating CD4+ T cells and have persistent, chronic immune activation (referred to as residual immune dysregulation syndrome (RIDS)), possibly leading to increased comorbid conditions such as age-associated diseases, inflammatory bowel disease, neurocognitive disorders, cardiovascular diseases, metabolic syndrome, bone abnormalities, and non-HIV-associated cancers, a phenomenon often referred to as “inflammaging”. Among the different plausible factor, one possibility would be the presence of soluble gp120 (sgp120) in the plasma and tissues of PLWH. Indeed, sgp120 was associated to HIV-1-induced immune dysfunction in a number of studies [4-7, 49]. sgp120 has been reported to exert proinflammatory activities, as binding of gp120 to CD4 on the surface of monocytes, macrophages, T cells and dendritic cells has been found to induce the production of cytokines, including interleukin 6 (IL-6), interleukin 10 and IL-1β, interferon α and γ, and tumor necrosis factor (TNF) α).

Notably, gp120 shed from productively infected cells has been shown to interact with CD4 present on uninfected bystander CD4+ T cells [15, 16]. This interaction leads to exposure of CD4-induced Env epitopes and sensitization of uninfected bystander CD4+ T cells to antibody-dependent cellular cytotoxicity (ADCC) mediated by HIV-positive plasma [16, 17]. Within HIV-positive plasma, antibodies targeting conserved CD4-induced gp120 cluster A epitopes have been shown to elicit potent ADCC activity against sgp120-coated cells [15, 18]. Antibodies recognizing the gp120 inner-domain cluster A region have been found to be responsible for most of the ADCC activity exhibited by chronically HIV-1-infected individuals, provided that the epitopes recognized by them are exposed [19].

A major problem to confirm or infirm the role of sgp120 in chronic immune activation, is the difficulty in measuring this antigen in biological fluids such as plasma, serum, cervicovaginal fluids, cerebrospinal fluids, etc.). ELISAs allowing the detection gp120 were reported. However, it remains unclear whether these assays can specifically detect the sgp120 over the gp120 among the trimeric Env present on HIV-1 viral particles. Notably, some of these assays require an inactivation step with lysis/disruption buffer that could dissociate the gp120 from the trimeric Env present on HIV-1 virions [11]. Others assays also exclusively rely on CD4-induced (CD4i) non-neutralizing antibodies (nnAbs) to detect and reveal the presence of gp120 [7]. These antibodies are easily elicited during HIV-1 infection and largely present in the plasma and biological fluids of infected individuals [34,35]. Therefore, the presence of these nnAbs in biological fluids has the potential to reduce the sensitivity of such ELISA assays.

There is thus a need for the development of novel assays and reagents for the detection of sgp120 in biological fluids.

The present description refers to a number of documents, the content of which is herein incorporated by reference in their entirety.

### SUMMARY

In various aspects and embodiments, the present disclosure provides the following items 1 to 55:
1. A method for detecting the presence of soluble gp120 (sgp120) in a sample comprising:
   (a) contacting the sample with a first sgp120-binding molecule that specifically binds to an epitope within the N-terminal 7-stranded or 8-stranded β-sandwich structure, preferably an epitope formed by residues 31-40, 42-43, 45, 84-87, 224, 244-246, and 491 of gp120, thereby forming a first complex between the first sgp120-binding molecule and sgp120 if sgp120 is present in the sample;
   (b) contacting the sample of (a) with a detection agent comprising:
      (i) a first detection agent comprising a second sgp120-binding molecule that specifically binds to an epitope within the constant region 1 and 2 (C1-C2) portion of the cluster A region of gp120, preferably an epitope formed by residues 51-54, 56, 58-61, 103, 106-107, 110, 114, 217, and 219-221 of gp120, conjugated to a third sgp120-binding molecule that specifically binds to a CD4-binding domain of gp120;
      (ii) a second detection agent comprising a fourth sgp120-binding molecule that specifically binds to an epitope within the bridging sheet of the Co-Receptor Binding Site (CoRBS), preferably an epitope formed by residues 119-122, 200, 202-205, 326-327, 369, 419-423 and 432-437 of gp120, conjugated to the third sgp120-binding molecule;
      (iii) a third detection agent comprising a fifth sgp120-binding molecule that specifically binds to an epitope formed by residues 97, 124, 198, 275-276, 278-283, 355, 365-368, 370-371, 425-427, 430, 455-463, 469 and 471-476 of gp120; or
      (iv) a combination of (i) to (iii); and
   (c) detecting the presence of a second complex between the detection agent and the first complex, wherein the presence of the second complex is indicative that sgp120 is present in the sample.
2. The method of item 1, wherein the first sgp120-binding molecule is an antibody or an antigen-binding fragment thereof, preferably antibody clone C11 or an antibody or an antigen-binding fragment thereof that competes with antibody clone C11.
3. The method of item 2, wherein the first sgp120-binding molecule is an antibody or an antigen-binding fragment thereof comprising the complementarity determining regions (CDRs) of antibody clone C11, DH677.3, L9-i2 or N12-i3.
4. The method of item 3, wherein the first sgp120-binding molecule is antibody clone C11, L9-i2 or N12-i3, preferably clone C11.
5. The method of any one of items 1 to 4, wherein the second sgp120-binding molecule is an antibody or an antigen-binding fragment thereof, preferably antibody clone A32 or an antibody or an antigen-binding fragment thereof that competes with antibody clone A32.
6. The method of item 5, wherein the second sgp120-binding molecule is an antibody or an antigen-binding fragment thereof comprising the complementarity determining regions (CDRs) of antibody clone A32, L9-i1 or N5-i5.
7. The method of item 6, wherein the second sgp120-binding molecule is antibody clone A32, L9-i1 or N5-i5, preferably clone A32.
8. The method of any one of items 1 to 7, wherein the fourth sgp120-binding molecule is an antibody or an antigen-binding fragment thereof, preferably antibody clone 17b or an antibody or an antigen-binding fragment thereof that competes with antibody clone 17b.
9. The method of item 8, wherein the fourth sgp120-binding molecule is an antibody or an antigen-binding fragment thereof comprising the complementarity determining regions (CDRs) of antibody clone 17b, X5, L9-i3, N5-i1, N5-i3, N5-i4, N5-i8, N10-i1.1, N10-i5.3, N12-i1, N12-i2, N12-i4, N12-i5, N12-i7, N12-i8, N12-i10, N12-i17, N12-i18, or N12-i9.
10. The method of item 9, wherein the fourth sgp120-binding molecule is antibody clone 17b, X5, L9-i3, N5-i1, N5-i3, N5-i4, N5-i8, N10-i1.1, N10-i5.3, N12-i1, N12-i2, N12-i4, N12-i5, N12-i7, N12-i8, N12-i10, N12-i17, N12-i18, or N12-i9, preferably antibody clone 17b or X5, more preferably antibody clone 17b.
11. The method of any one of items 1 to 10, wherein the fifth sgp120-binding molecule is an antibody or an antigen-binding fragment thereof, preferably antibody clone N6 or an antibody or an antigen-binding fragment thereof that competes with antibody clone N6.
12. The method of item 11, wherein the fifth sgp120-binding molecule is an antibody or an antigen-binding fragment thereof comprising the complementarity determining regions (CDRs) of antibody clone N6, N49P6, N49P7 or N49P11.
13. The method of item 12, wherein the fifth sgp120-binding molecule is antibody clone N6, N49P6, N49P7 or N49P11, preferably antibody clone N6b.
14. The method of any one of items 1 to 13, wherein the third sgp120-binding molecule is a polypeptide comprising domains 1 and 2 of human CD4 receptor.
15. The method of any one of items 1 to 14, wherein the third sgp120-binding molecule is conjugated to the second and/or fourth sgp120-binding molecule via a polypeptide linker.
16. The method of any one of items 1 to 15, wherein the detection agent comprises the first and second detection agents.
17. The method of any one of items 1 to 15, wherein the detection agent comprises the third detection agent.
18. The method of any one of items 1 to 17, wherein the first sgp120-binding molecule is attached to a solid support, such as a plate.
19. The method of any one of items 1 to 17, wherein the first, second and/or third detection agent is conjugated to a detectable label.
20. The method of item 19, wherein the detectable label is an enzyme.
21. The method of item 20, wherein detecting the presence of a second complex comprises adding a substrate for the enzyme, and detecting a product generated from the substrate by the enzyme.
22. The method of item 20 or 21, wherein the enzyme is horseradish peroxidase (HRP).
23. The method of any one of items 1 to 22, wherein the sample is a biological sample.
24. The method of item 23, wherein the biological sample is blood or plasma.
25. A kit comprising:
   (a) a first soluble gp120 (sgp120)-binding molecule that specifically binds to an epitope within the N-terminal 7-stranded or 8-stranded β-sandwich structure, preferably an epitope formed by residues 31-40, 42-43, 45, 84-87, 224, 244-246, and 491 of gp120;
   (b) a detection agent comprising:
      (i) a first detection agent comprising a second sgp120-binding molecule that specifically binds to an epitope within the constant region 1 and 2 (C1-C2) portion of the cluster A region of gp120, preferably an epitope formed by residues 51-54, 56, 58-61, 103, 106-107, 110, 114, 217, and 219-221 of gp120, conjugated to a third sgp120-binding molecule that specifically binds to a CD4-binding domain of gp120;
      (ii) a second detection agent comprising a fourth sgp120-binding molecule that specifically binds to an epitope within the bridging sheet of the Co-Receptor Binding Site (CoRBS), preferably an epitope formed by residues 119-122, 200, 202-205, 326-327, 369, 419-423 and 432-437 of gp120, conjugated to the third sgp120-binding molecule;
      (iii) a third detection agent comprising a fifth sgp120-binding molecule that specifically binds to an epitope formed by residues 97, 124, 198, 275-276, 278-283, 355, 365-368, 370-371, 425-427, 430, 455-463, 469 and 471-476 of gp120; or
      (iv) a combination of (i) to (iii).
26. The kit of item 25, wherein the first sgp120-binding molecule is an antibody or an antigen-binding fragment thereof, preferably antibody clone C11 or an antibody or an antigen-binding fragment thereof that competes with antibody clone C11.
27. The kit of item 26, wherein the first sgp120-binding molecule is an antibody or an antigen-binding fragment thereof comprising the complementarity determining regions (CDRs) of antibody clone C11, DH677.3, L9-i2 or N12-i3.
28. The kit of item 27, wherein the first sgp120-binding molecule is antibody clone C11, L9-i2 or N12-i3, preferably clone C11.
29. The kit of any one of items 25 to 28, wherein the second sgp120-binding molecule is an antibody or an antigen-binding fragment thereof, preferably antibody clone A32 or an antibody or an antigen-binding fragment thereof that competes with antibody clone A32.
30. The kit of item 29, wherein the second sgp120-binding molecule is an antibody or an antigen-binding fragment thereof comprising the complementarity determining regions (CDRs) of antibody clone A32, L9-i1 or N5-i5.
31. The kit of item 30, wherein the second sgp120-binding molecule is antibody clone A32, L9-i1 or N5-i5, preferably clone A32.
32. The kit of any one of items 25 to 31, wherein the fourth sgp120-binding molecule is an antibody or an antigen-binding fragment thereof, preferably antibody clone 17b or an antibody or an antigen-binding fragment thereof that competes with antibody clone 17b.
33. The kit of item 32, wherein the fourth sgp120-binding molecule is an antibody or an antigen-binding fragment thereof comprising the complementarity determining regions (CDRs) of antibody clone 17b, X5, L9-i3, N5-i1, N5-i3, N5-i4, N5-i8, N10-i1.1, N10-5.3, N12-i1, N12-i2, N12-i4, N12-i5, N12-i7, N12-i8, N12-i10, N12-i17, N12-i18, or N12-i9.
34. The kit of item 33, wherein the fourth sgp120-binding molecule is antibody clone 17b, X5, L9-i3, N5-i1, N5-i3, N5-i4, N5-i8, N10-i1.1, N10-i5.3, N12-i1, N12-i2, N12-i4, N12-i5, N12- i7, N12-i8, N12-i10, N12-i17, N12-i18, or N12-i9, preferably antibody clone 17b or X5, more preferably antibody clone 17b.
35. The kit of anyone of items 25 to 34, wherein the fifth sgp120-binding molecule is an antibody or an antigen-binding fragment thereof, preferably antibody clone N6 or an antibody or an antigen-binding fragment thereof that competes with antibody clone N6.
36. The kit of item 35, wherein the fifth sgp120-binding molecule is an antibody or an antigen-binding fragment thereof comprising the complementarity determining regions (CDRs) of antibody clone N6, N49P6, N49P7 or N49P11.
37. The kit of item 36, wherein the fifth sgp120-binding molecule is antibody clone N6, N49P6, N49P7 or N49P11, preferably antibody clone N6b.
38. The kit of any one of items 25 to 37, wherein the third sgp120-binding molecule is a polypeptide comprising domains 1 and 2 of human CD4 receptor.
39. The kit of any one of items 25 to 38, wherein the third sgp120-binding molecule is conjugated to the second and/or fourth sgp120-binding molecule via a polypeptide linker.
40. The kit of any one of items 25 to 39, wherein the detection agent comprises the first and second detection agents.
41. The kit of any one of items 25 to 40, wherein the detection agent comprises the third detection agent.
42. The kit of any one of items 25 to 41, further comprising a solid support, such as a plate.
43. The kit of item 42, wherein the first sgp120-binding molecule is attached to the solid support.
44. The kit of any one of items 25 to 43, wherein the first, second and/or third detection agent is conjugated to a detectable label.
45. The kit of item 44, wherein the detectable label is an enzyme.
46. The kit of item 45, wherein the enzyme is horseradish peroxidase (HRP).
47. The kit of item 45, further comprising a substrate for the enzyme.
48. The kit of any one of items 25 to 47, which further comprises instructions for detecting the presence of sgp120 in a sample according to the method of any one of items 1 to 24.
49. A method of identifying an HIV-infected individual suffering from or at risk of suffering from HIV-associated inflammaging or chronic immune activation (residual immune dysregulation syndrome, RIDS), the method comprising performing the method defined in any one of items 1 to 24 on a biological sample from the HIV-infected individual, wherein the presence of sgp120 in the sample is indicative that the individual suffers from or is at risk of suffering from HIV-associated inflammaging or chronic immune activation.
50. A method of treating HIV-associated inflammaging or residual immune dysregulation syndrome (RIDS) in an HIV-infected individual, the method comprising administering an effective amount of an HIV-1 attachment inhibitor to the HIV-infected individual suffering from RIDS identified by the method of item 49.
51. The method of item 50, wherein the HIV-1 attachment inhibitor is temsavir or fostemsavir.
52. An HIV-1 attachment inhibitor for use in the treatment of HIV-associated inflammaging or residual immune dysregulation syndrome (RIDS) in an HIV-infected individual, wherein the HIV-infected individual suffering from HIV-associated inflammaging or RIDS is identified by the method of item 49.
53. The HIV-1 attachment inhibitor for use according to item 52, wherein the HIV-1 attachment inhibitor is temsavir or fostemsavir.
54. Use of an HIV-1 attachment inhibitor for the manufacture of a medicament for the treatment of HIV-associated inflammaging or residual immune dysregulation syndrome (RIDS) in an HIV-infected individual, wherein the HIV-infected individual suffering from HIV-associated inflammaging or RIDS is identified by the method of item 49.
55. The use according to item 54, wherein the HIV-1 attachment inhibitor is temsavir or fostemsavir.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts a schematic of the sandwich ELISA used to detect soluble gp120. ELISA plates are coated with the C11 Ab and then incubated with a biological sample such as diluted plasma. The C11 Abs is used as a bait to grab the sgp120 present in the plasma. Following washing steps, the N6-HRP or the combination of 17b-sCD4-HRP/A32-sCD4-HRP is added to the plate to attach to sgp120. Following washing steps, an HRP substrate solution is added to initiate the reaction. Light emission is measured with a luminometer.

FIGS. 2A and 2B are graphs showing the detection of soluble gp120 in plasma from HIV-1-infected individuals using C11 in combination with N6-HRP or VRC01-HRP. Soluble gp120 was detected in the plasma from 50 HIV-1-infected individuals and 20 HIV-negative individuals using the CD4-binding site Abs N6-HRP (left) or VRC01-HRP (right) in the detection antibody solution. The dashed line represents the positivity threshold.

FIG. 3 is a graph showing a standard curve for the detection of soluble gp120 using N6-HRP or Ab-sCD4-HRP. Standard curves were established with 2-fold serial dilution of monomeric gp120 in the plasma of HIV uninfected donor.

FIGS. 4A and B are graphs showing the detection of soluble gp120 in plasma from ART-treated individuals using C11 in combination with N6-HRP or the Ab-sCD4-HRP. Soluble gp120 was detected in the plasma from 94 chronically-infected ART-treated individuals and 20 HIV-negative individuals using the CD4-binding site Abs N6-HRP (left) or the combination of 17b-sCD4-HRP/A32-sCD4-HRP (right) in the detection antibody solution. The dashed line represents the positivity threshold.

FIG. 5A shows the schematic of the sgp120 ELISA assay using the C11 and N6-HRP antibodies.

FIG. 5B shows the standard curve obtained with this assay using reference recombinant sgp120 (n=4). Statistical analysis was performed using simple linear regression.

FIGS. 6A-B show the detection of soluble glycoprotein 120 (sgp120) in people living with human immunodeficiency virus (HIV) with undetectable viremia is associated with inflammation. Representative stratification is shown for 157 people living with HIV, based on levels of sgp120 (FIG. 6A) and interleukin 6 (IL-6) (FIG. 6A). Statistical analysis was performed using Mann-Whitney U tests. *P<0.05; **P<0.01; ***P<0.001. Abbreviations: HIV-, HIV negative; HIV+, HIV positive; NS, not significant; RU, relative units.

FIGS. 7A-D show that anti-cluster A antibodies are inversely correlated with CD4⁺ T-cell counts in people living with human immunodeficiency virus (PLWH) presenting with high levels of soluble glycoprotein 120 (sgp120). Correlations between CD4⁺ T-cell counts and anti-cluster A antibody levels are shown for 386 PLWH, stratified by sgp120 levels. Correlations are shown for the total study population (FIG. 7A) and for PLWH with undetectable sgp120 (FIG. 7B), low levels of sgp120 (sgp120_{low}) (FIG. 7C), or high levels of sgp120 (sgp120_{high}) (FIG. 7D). Levels of anti-cluster A antibodies were log₂ transformed. Univariable and multivariable linear regressions were performed, with the beta parameters representing the mean predicted change in absolute CD4⁺ cell counts for each 1-log₂ increase in titers of anti-cluster A antibodies. Multivariable models are adjusted for age, sex, ethnicity, smoking status, duration of antiretroviral therapy, nadir CD4⁺ cell counts, and levels of anti-CD4 binding site antibodies. Abbreviations: CI, confidence interval; RU, relative units.

FIGS. 8A-D show that anti-cluster A antibodies are inversely correlated with CD4:CD8 ratio in PLWH presenting with high levels of soluble glycoprotein 120 (sgp120). Correlations between the CD4:CD8 ratio and anti-cluster A antibody levels upon stratification of 386 PLWH by sgp120 levels. Correlations are depicted for the total study population (FIG. 8A) and for PLWH with undetectable sgp120 (FIG. 8B), low levels of sgp120 (sgp120_{low}) (FIG. 8C), or high levels of sgp120 (sgp120_{high}) (FIG. 80). Levels of anti-cluster A antibodies were log₂ transformed. Univariable and multivariable linear regressions were performed, with the beta parameters representing the mean predicted change in CD4:CD8 ratio for each 1-log₂ increase in titers of anti-cluster A antibodies. Multivariable models are adjusted for age, sex, ethnicity, smoking status, duration of antiretroviral therapy, nadir CD4⁺ cell counts, and levels of anti-CD4 binding site antibodies. Abbreviations: CI, confidence interval; RU, relative units.

FIGS. 9A-C show that the combination of soluble glycoprotein 120 (sgp120) levels and anti-cluster A antibodies correlates positively with subclinical cardiovascular disease. Associations are displayed between the size of coronary artery plaque volume and sgp120 levels (FIG. 9A), anti-cluster A antibodies (FIG. 9B), and the multiplicative combination of both (FIG. 9C) in people living with human immunodeficiency virus who are positive for sgp120 and have detectable subclinical cardiovascular disease. Values for sgp120, anti-cluster A antibodies, and total plaque volume (mm³) were log₂ transformed. Univariable and multivariable linear regressions were performed, with the beta parameters representing the mean predicted change in log total plaque volume for each 1-log₂ increase in the exposure. Multivariable models are adjusted for age, sex, smoking status, low- or high-density lipoproteins, diabetes mellitus, hypertension, alcohol use disorder, intravenous drug use, and duration of antiretroviral therapy. Abbreviations: CI, confidence interval; RU, relative units.

FIGS. 10A-D show a longitudinal analysis of soluble glycoprotein 120 (sgp120) and some inflammatory markers. Levels of sgp120 (FIG. 10A), interleukin 6 (IL-6) (FIG. 10B), tumor necrosis factor (TNF) a (FIG. 10C), and soluble CD163 (sCD163) (FIG. 100) were measured over time for 9 participants with detectable levels of sgp120. Dashed line in FIG. 10A represents seropositivity threshold established in plasma samples from uninfected participants. Abbreviation: RU, relative units.

FIGS. 11A-B show a longitudinal analysis of anti-cluster A antibodies and CD4⁺ T-cell counts. Levels of anti-cluster A antibodies (FIG. 11A) and CD4⁺ T-cell counts (FIG. 11B) were measured overtime for 9 participants with detectable levels of soluble glycoprotein 120 (sgp120). Abbreviation: RU, relative units.

FIG. 12A depicts the amino acid sequences of the heavy and light chains of anti-gp120 antibody clone C11, with the CDR1, CDR2 and CDR3 underlined (Chothia numbering).

FIG. 12B depicts the amino acid sequences of the heavy and light chains of anti-gp120 antibody clone n12-i3.

FIG. 12C depicts the amino acid sequences of the heavy and light chain of the anti-gp120 A32-sCD4 construct. The heavy chain of antibody clone A32 (bold) is fused at its N-terminal end to a soluble CD4 polypeptide (italics) through a linker. The CDR1, CDR2 and CDR3 of the heavy and light chains are underlined (Chothia numbering).

FIG. 12D depicts the amino acid sequences of the heavy and light chains of anti-gp120 antibody clone 2.2C.2.

FIG. 12E depicts the amino acid sequences of the heavy and light chains of anti-gp120 antibody clone n5-i5.

FIG. 12F depicts the amino acid sequences of the heavy and light chain of the anti-gp120 17b-sCD4 construct. The heavy chain of antibody clone 17b (bold) is fused at its N-terminal end to a soluble CD4 polypeptide (italics) through a linker. The CDR1, CDR2 and CDR3 of the heavy and light chains are underlined (Chothia numbering).

FIG. 12G depicts the amino acid sequences of the heavy and light chains of anti-gp120 antibody clone 412d.

FIG. 12H depicts the amino acid sequences of the heavy and light chains of anti-gp120 antibody clone 412d.

FIG. 12I depicts the amino acid sequences of the heavy and light chains of anti-gp120 antibody clone E51.

FIG. 12J depicts the amino acid sequences of the heavy and light chains of anti-gp120 antibody clone n12-i2.

FIG. 12K depicts the amino acid sequences of the heavy and light chains of anti-gp120 antibody clone X5.

FIG. 12L depicts the amino acid sequences of the heavy and light chain of anti-gp120 antibody clone N6. The CDR1, CDR2 and CDR3 of the heavy and light chains are underlined (Chothia numbering).

FIG. 12M depicts the amino acid sequences of the heavy and light chains of anti-gp120 antibody clone N49P6.

FIG. 12N depicts the amino acid sequences of the heavy and light chains of anti-gp120 antibody clone N49P7.

FIG. 12O depicts the amino acid sequences of the heavy and light chains of anti-gp120 antibody clone N60P1.1.

FIG. 12P depicts the amino acid sequences of the heavy and light chains of anti-gp120 antibody clone N60P23.

FIG. 12Q depicts the amino acid sequences of the heavy and light chains of anti-gp120 antibody clone N49P9.

FIG. 12R depicts the amino acid sequences of the heavy and light chains of anti-gp120 antibody clone N49P9.1.

FIG. 12S depicts the amino acid sequences of the heavy and light chains of anti-gp120 antibody clone N49P9.3.

FIG. 12T depicts the amino acid sequences of the heavy and light chains of anti-gp120 antibody clone N49P11.

FIG. 13A depicts the amino acid sequence of gp120 from HIV reference strain HXB2 (SEQ ID NO:43).

FIG. 13B depicts the amino acid sequence of human CD4 (SEQ ID NO:44), with the D1 and D2 domains (residues 26 to 208) in bold underlined.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in biology, virology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have developed a sensitive assay to detect soluble gp120 in biological fluids that does not involved an inactivation step with lysis/disruption buffer, and whose sensitivity is not negatively affected by the presence of non-neutralizing antibodies (nnAbs) in the biological sample tested.

The present disclosure provides a method for detecting the presence of soluble gp120 (sgp120) in a sample comprising:
(a) contacting the sample with a capture agent comprising a first sgp120-binding molecule that specifically binds to an epitope within the N-terminal 8-stranded β-sandwich structure of gp120, thereby forming a first complex between the first sgp120-binding molecule and sgp120 if sgp120 is present in the sample;
(b) contacting the sample of (a) with a detection agent comprising:
   (i) a first detection agent comprising a second sgp120-binding molecule that specifically binds to an epitope within the constant region 1 and 2 (C1-C2) portion of the cluster A region of gp120, conjugated to a third sgp120-binding molecule that specifically binds to a CD4-binding domain of gp120;
   (ii) a second detection agent comprising a fourth sgp120-binding molecule that specifically binds to an epitope within the bridging sheet of the Co-Receptor Binding Site (CoRBS) of gp120, conjugated to the third sgp120-binding molecule;
   (iii) a third detection agent comprising a fifth sgp120-binding molecule that is antibody clone N6 or a binding molecule that competes with antibody clone N6 for binding to gp120; or
   (iv) a combination of (i) to (iii); and
      (c) detecting the presence of a second complex between the detection agent and the first complex, wherein the presence of the second complex is indicative that sgp120 is present in the sample.

The present disclosure provides a kit, e.g., a kit for detecting the presence of sgp120 in a sample, comprising:
(a) a capture agent comprising a first soluble gp120 (sgp120)-binding molecule that specifically binds to an epitope within the N-terminal 8-stranded β-sandwich structure of gp120;
(b) a detection agent comprising:
   (i) a first detection agent comprising a second sgp120-binding molecule that specifically binds to an epitope within the constant region 1 and 2 (C1-C2) portion of the cluster A region of gp120, conjugated to a third sgp120-binding molecule that specifically binds to a CD4-binding domain of gp120;
   (ii) a second detection agent comprising a fourth sgp120-binding molecule that specifically binds to an epitope within the bridging sheet of the Co-Receptor Binding Site (CoRBS) of gp120, conjugated to the third sgp120-binding molecule;
   (iii) a third detection agent comprising a fifth sgp120-binding molecule that is antibody clone N6 or a binding molecule that competes with antibody clone N6 for binding to gp120; or
(iv) a combination of (i) to (iii).

As used herein, the term “sgp120-binding molecule” refers to any molecule capable of binding to the specified domain or epitope. The term “binding molecule” encompasses antibodies, antibody fragments and non-antibody binding agents, for example antibody mimetics such as those described in Yu et al. (2017) Annu Rev Anal Chem 10(1):293-320. Thus, the sgp120-binding molecules defined herein may be ligands of gp120 (natural or synthetic), antibodies, antibody fragments, antibody mimetics, adnectins, affibodies, affilins, affimers, affitins, alphabodies, anticalins, aptamers, armadillo repeat protein-based scaffolds, atrimers, avimers, DARPins, fynomers, knottins, Kunitz domain peptides, monobodies, and nanofitins.

In embodiments, the first, second, fourth and/or fifth sgp120-binding molecules are antibodies or antigen-binding fragments thereof. In further embodiments, the first, second, fourth and fifth sgp120-binding molecules are antibodies or antigen-binding fragments thereof. The terms “antibody” and “antibodies” refer to naturally occurring forms including monoclonal or polyclonal antibodies, or recombinant antibodies such as chimeric antibodies or humanized antibodies. The term “antibody fragment” includes, for example, F(ab), F(ab′)₂, Fv, single chain antibodies or diabodies. In an embodiment, the antibody is naturally-occurring, full-length human antibody.

In an embodiment, the first sgp120-binding molecule is antibody clone C11 or a sgp120-binding molecule (e.g., an antibody or an antigen-binding fragment thereof) that competes with antibody clone C11 for binding to sgp120. As used herein, the expression “competes with” means that the sgp120-binding molecule inhibits or reduces the binding of antibody clone C11 to sgp120, which indicates that the sgp120-binding molecule binds to a domain or epitope in sgp120 that overlaps with the domain or epitope bound by antibody clone C11. Examples of sgp120-binding molecules that compete with antibody clone C11 include antibody clones L9-i2 and N12-i3 (Guan, Yongjun et al. “Diverse specificity and effector function among human antibodies to HIV-1 envelope glycoprotein epitopes exposed by CD4 binding.” Proceedings of the National Academy of Sciences of the United States of America vol. 110,1 (2013): E69-78. doi:10.1073/pnas.1217609110), as well as antibodies CH54, CH55 and DH677.3 (Tolbert et al., mBio. 2020 May-Jun; 11(3): e00208-20). In an embodiment, the first sgp120-binding molecule specifically binds to an epitope located within the N-terminal 7-stranded or 8-stranded β-sandwich structure of gp120. In an embodiment, the first sgp120-binding molecule specifically binds to an epitope formed by residues 31-40, 42-43, 45, 84-87, 224, 244-246, and 491 of gp120. Residue numbering is based on the sequence of gp120 from reference HIV strain HXB2 (FIG. 13A).

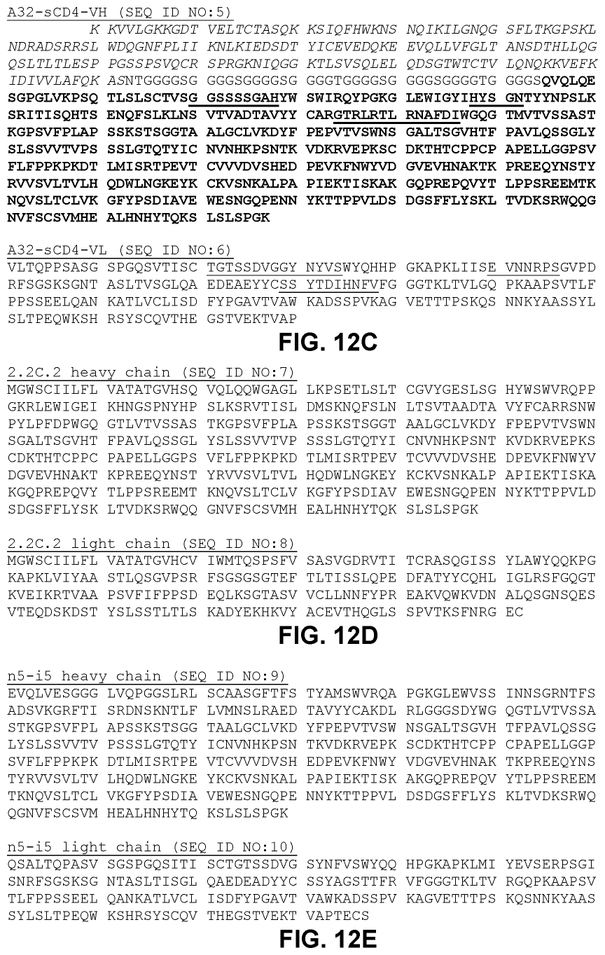

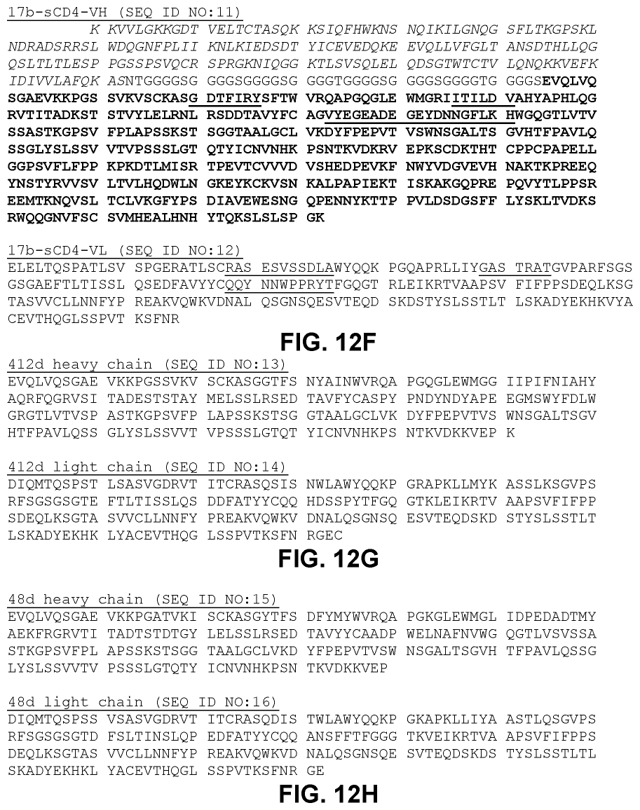

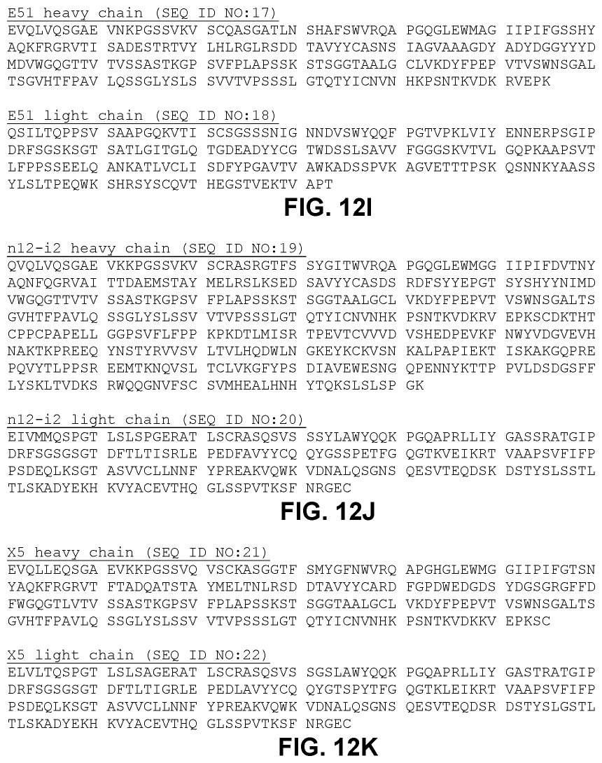

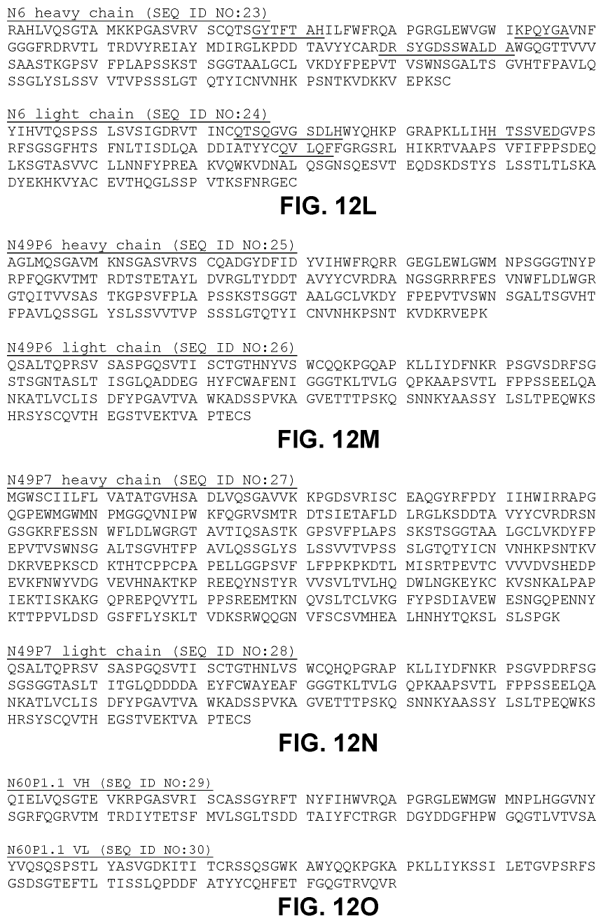

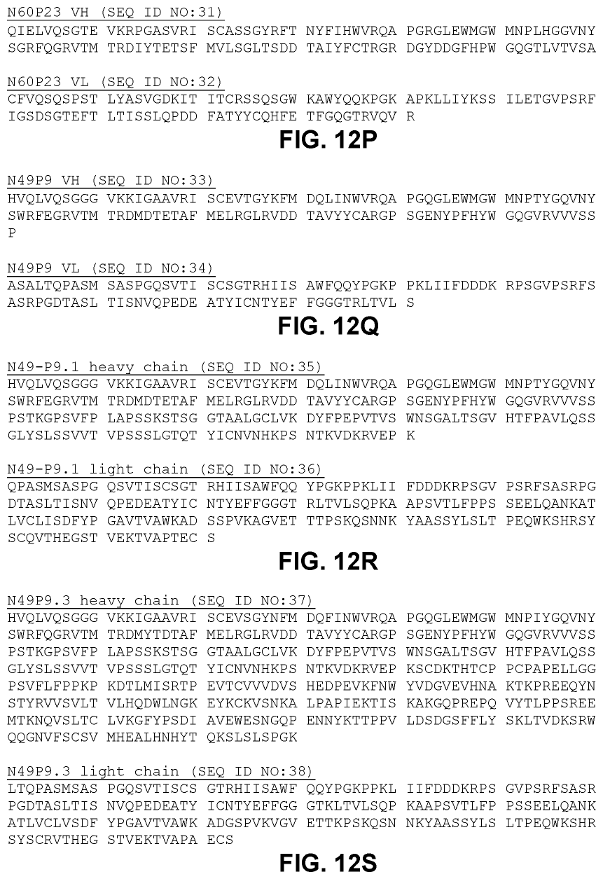

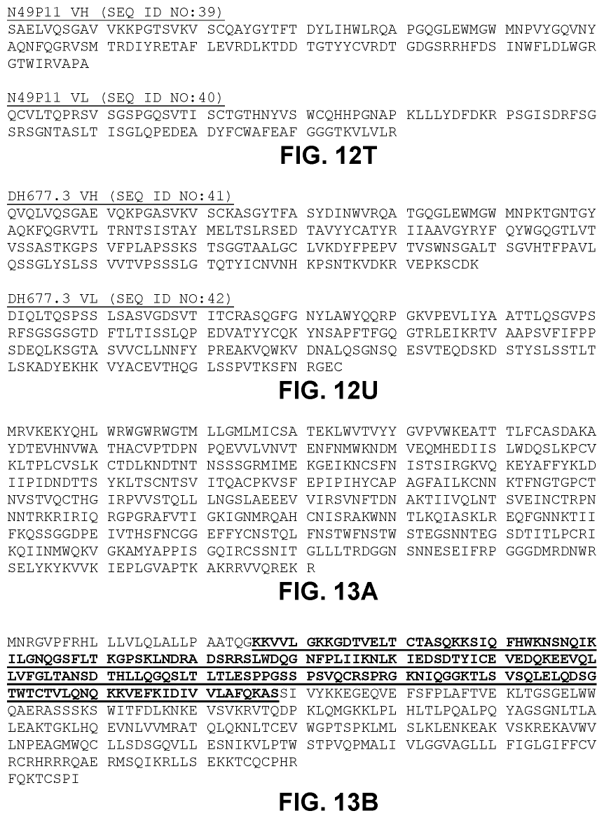

In an embodiment, the first sgp120-binding molecule is an antibody or an antigen-binding fragment thereof comprising the complementarity determining regions (CDRs) of antibody clone C11, L9-i2, DH677.3 or N12-i3, preferably C11. In an embodiment, the first sgp120-binding molecule is an antibody or an antigen-binding fragment thereof comprising the variable regions of antibody clone C11, L9-i2, DH677.3 or N12-i3, preferably C11. In a further embodiment, the first sgp120-binding molecule is antibody clone C11, L9-i2, DH677.3 or N12-i3, preferably C11. The amino acid sequences of the heavy and light chains of antibody clone C11 are depicted in FIG. 12A, the amino acid sequences of the heavy and light chains of antibody clone N12-i3 are depicted in FIG. 12B, and the amino acid sequences of the heavy and light chains of antibody clone DH677.3 are depicted in FIG. 12U.

In an embodiment, the second sgp120-binding molecule is antibody clone A32 or a sgp120-binding molecule (e.g., an antibody or an antigen-binding fragment thereof) that competes with antibody clone A32 for binding to sgp120. Examples of sgp120-binding molecules that compete with antibody clone A32 include antibody clones L9-i1, N5-i5, N60-i3, 2.2c (Guan, Yongjun et al. “Diverse specificity and effector function among human antibodies to HIV-1 envelope glycoprotein epitopes exposed by CD4 binding.” Proceedings of the National Academy of Sciences of the United States of America, vol. 110, 1 (2013): E69-78. doi:10.1073/pnas.1217609110; Tolbert et al., “Structural Basis for Epitopes in the gp120 Cluster A Region that Invokes Potent Effector Cell Activity”, Viruses. 2019 January; 11(1): 69). In an embodiment, the second sgp120-binding molecule specifically binds to an epitope formed by residues within the following regions: 51-54, 56, 58-61, 68-80, 103, 106-107, 110, 113-114, 217, and 219-221 of gp120.

In an embodiment, the second sgp120-binding molecule is an antibody or an antigen-binding fragment thereof comprising the complementarity determining regions (CDRs) of antibody clone A32, N60-i3, L9-i1, N60-i3, 2.2c, 2.2c.2 or N5-i5, preferably A32. In an embodiment, the second sgp120-binding molecule is an antibody or an antigen-binding fragment thereof comprising the variable regions of antibody clone A32, L9-i1, 2.2c, 2.2.c.2 or N5-i5, preferably A32. In a further embodiment, the second sgp120-binding molecule is antibody clone A32, L9-i1, 2.2c, 2.2.c.2 or N5-i5, preferably A32. The amino acid sequences of the heavy and light chains of antibody clone A32 are depicted in FIG. 12C, the amino acid sequences of the heavy and light chains of antibody clone 2.2c.2 are depicted in FIG. 12D, and the amino acid sequences of the heavy and light chains of antibody clone N5-i5 are depicted in FIG. 12E.

The third sgp120-binding molecule is a molecule that specifically binds to a CD4-binding domain of gp120. Such molecules are well known in the art and include “small CD4 mimetic” or “CD4mc” such as NBD-556, NBD-557, DMJ-1-228, JP-III-48, M48U1 and BNM-III-170. CD4mc are also disclosed in PCT publications Nos. WO 2013/090696 and WO 2020/028482. Molecules that specifically bind to a CD4-binding domain of gp120 also includes peptides and polypeptides, such as peptides or polypeptides derived from the human CD4 receptor. In an embodiment, the third sgp120-binding molecule is a soluble human CD4 peptide or polypeptide (sCD4). In a further embodiment, the soluble human CD4 peptide or polypeptide comprises a sequence having at least 70%, 75%, 80%, 85%, 90% or 95% with the sequence of the D1 and D2 domains of human CD4, e.g., residues 26 to 208, FIG. 13B). In a further embodiment, the soluble human CD4 peptide or polypeptide comprises residues 26 to 208 of human CD4 (Richard et al., mBio, Vol. 12(5): e1405-21).

In an embodiment, the third sgp120-binding molecule is conjugated to the second and/or fourth sgp120-binding molecules through a linker, preferably a peptide or polypeptide linker. In an embodiment, the linker has a length of 20 to 100 amino acids, for example 20 to 80, 20 to 60, 30 to 50, 35 to 45, or 40 amino acids. In an embodiment, the linker comprises glycine (Gly), serine (Ser) and/or threonine (Thr) residues. In further embodiments, the linker comprises glycine, serine and threonine residues. In further embodiments, the linker comprises [Gly₄-Ser] and/or [Gly₄-Thr] motifs, for example [Gly₄-Ser]ₘ and [Gly₄-Thr]ₙ, motifs, wherein m is an integer from 3 to 10, preferably from 4 to 8 (e.g., 6), and n is an integer from 1 to 4, preferably 1 to 3 (e.g., 2).

In an embodiment, the second and/or fourth sgp120-binding molecules are antibodies or antigen-binding fragments thereof, and wherein the third sgp120-binding molecule is conjugated to the N-terminal end of the heavy chains of the antibodies or antigen-binding fragments thereof.

In an embodiment, the fourth sgp120-binding molecule is antibody clone 17b or a sgp120-binding molecule (e.g., an antibody or an antigen-binding fragment thereof) that competes with antibody clone 17b for binding to sgp120. Examples of sgp120-binding molecules that compete with antibody clone 17b include antibody clones X5, 412d, 48d, E51, L9-i3, N5-i1, N5-i3, N5-i4, N5-i8, N10-i1.1, N10-i5.3, N12-i1, N12-i2, N12-i4, N12-5, N12-i7, N12-i8, N12-i10, N12-i17, N12-i18, and N12-i9 (Guan, Yongjun et al. “Diverse specificity and effector function among human antibodies to HIV-1 envelope glycoprotein epitopes exposed by CD4 binding.” Proceedings of the National Academy of Sciences of the United States of America, vol. 110,1 (2013): E69-78. doi:10.1073/pnas.1217609110). In an embodiment, the fourth sgp120-binding molecule specifically binds to an epitope formed by residues within the following regions: 119-122, 200, 202-205, 326-327, 369, 419-423 and 432-437 of gp120.

In an embodiment, the fourth sgp120-binding molecule is an antibody or an antigen-binding fragment thereof comprising the complementarity determining regions (CDRs) of antibody clone 17b, X5, 412d, 48d, E51, L9-i3, N5-i1, N5-i3, N5-i4, N5-i8, N10-i1.1, N10-i5.3, N12-i1, N12-i2, N12-i4, N12-i5, N12-i7, N12-i8, N12-i10, N12-i17, N12-i18, or N12-i9, preferably 17b. In an embodiment, the fourth sgp120-binding molecule is an antibody or an antigen-binding fragment thereof comprising the variable regions of antibody clone 17b, X5, 412d, 48d, E51, L9-i3, N5-i1, N5-i3, N5-i4, N5-i8, N10-i1.1, N10-i5.3, N12-i1, N12-i2, N12-i4, N12-i5, N12-i7, N12-i8, N12-i10, N12-i17, N12-i18, or N12-i9, preferably 17b. In a further embodiment, the fourth sgp120-binding molecule is antibody clone 17b, X5, 412d, 48d, E51, L9-i3, N5-i1, N5-i3, N5-i4, N5-i8, N10-i1.1, N10-i5.3, N12-i1, N12-i2, N12-i4, N12-i5, N12-i7, N12-i8, N12-i10, N12-i17, N12-i18, or N12-i9, preferably 17b. The amino acid sequences of the heavy and light chains of antibody clone 17b are depicted in FIG. 12F, the amino acid sequences of the heavy and light chains of antibody clone 412d are depicted in FIG. 12G, and the amino acid sequences of the heavy and light chains of antibody clone 48d are depicted in FIG. 12H, the amino acid sequences of the heavy and light chains of antibody clone E51 are depicted in FIG. 12I, the amino acid sequences of the heavy and light chains of antibody clone N12-i2 are depicted in FIG. 12J, the amino acid sequences of the heavy and light chains of antibody clone X5 are depicted in FIG. 12K.

The fifth sgp120-binding molecule is antibody clone N6 or a sgp120-binding molecule (e.g., an antibody or an antigen-binding fragment thereof) that competes with antibody clone N6 for binding to sgp120. Antibody clone N6 is a CD4-binding site (CD4bs) antibody, and relative to most VRC01-class antibodies, it is characterized by strong binding to the conserved D loop of gp120, with no or minimal interaction with residues from the variable V5 loop, thereby tolerating mutations in the variable V5 loop (Huang et al., Immunity 45, 1108-1121, Nov. 15, 2016). Examples of CD4bs antibodies having similar binding characteristics as clone N6 include the N60 and N49 P antibody series, such as N60 P1.1, N60P23, N49P6, N49P7, N49P9, N49P9.1 or N49P11 (Sajadi et al., 2018, Cell 173, 1783-1795). In an embodiment, the fifth sgp120-binding molecule specifically binds to an epitope formed by residues 97, 124, 198, 275-276, 278-283, 355, 365-368, 370-371, 425-427, 430, 455-463, 469 and 471-476 of gp120.

In an embodiment, the fifth sgp120-binding molecule is an antibody or an antigen-binding fragment thereof comprising the complementarity determining regions (CDRs) of antibody clone N6, N60P1.1, N60P23, N49P6, N49P7, N49P9, N49P9.1, N49P9.3 or N49P11, preferably N6. In an embodiment, the fourth sgp120-binding molecule is an antibody or an antigen-binding fragment thereof comprising the variable regions of antibody clone N6, N60P1.1, N60P23, N49P6, N49P7, N49P9, N49P9.1, N49P9.3 or N49P11, preferably N6. In a further embodiment, the fourth sgp120-binding molecule is antibody clone N6, N60P1.1, N60P23, N49P6, N49P7, N49P9, N49P9.1, N49P9.3 or N49P11, preferably N6. The amino acid sequences of the heavy and light chains of antibody clone N6 are depicted in FIG. 12L, the amino acid sequences of the heavy and light chains of antibody clone N49P6 are depicted in FIG. 12M, the amino acid sequences of the heavy and light chains of antibody clone N49P7 are depicted in FIG. 12N, the amino acid sequences of the heavy and light chains of antibody clone N60P1.1 are depicted in FIG. 12O, the amino acid sequences of the heavy and light chains of antibody clone N60P23 are depicted in FIG. 12P, the amino acid sequences of the heavy and light chains of antibody clone N49P9 are depicted in FIG. 12Q, the amino acid sequences of the heavy and light chains of antibody clone N49P9.1 are depicted in FIG. 12R, the amino acid sequences of the heavy and light chains of antibody clone N49P9.3 are depicted in FIG. 12S, the amino acid sequences of the heavy and light chains of antibody clone N49P11 are depicted in FIG. 12T.

Relevant information concerning the antibody clones described herein may be found, e.g., in the HIV database (Los Alamos National Laboratory, HIV Molecular Immunology 2021, Editors: Elizabeth-Sharon David-Fung, Bette T. M. Korber, Christian Brander, Dan Barouch, Rob de Boer, Barton F. Haynes, Richard Koup, John P. Moore, Bruce D. Walker, and David I. Watkins. Publisher: Los Alamos National Laboratory, Theoretical Biology and Biophysics, Los Alamos, New Mexico. LA-UR-21-32446, https://www.hiv.lanl.gov/content/index), the RCSB Protein Data Bank (PDB) (H. M. Berman, J. Westbrook, Z. Feng, G. Gilliland, T. N. Bhat, H. Weissig, I. N. Shindyalov, P. E. Bourne. (2000) The Protein Data Bank Nucleic Acids Research, 28: 235-242, https://www.rcsb.org/), and the references cited above.

Based on the sequences of the heavy and light chains of the antibodies disclosed herein (see FIGS. 12A-12U), the skilled person would be able to easily identify the amino acid sequences corresponding to the CDRs and FRs of these antibodies based on common general knowledge in the field of antibodies. The sequences of the CDRs of representative antibodies are identified in FIGS. 12A, 12C, 12F, and 12L, but the skilled person would be able to easily identify the sequences of the CDRs of all other antibodies disclosed herein. The CDRs and FRs of antibodies may for example by identified using commonly available tools such as the abYsis tool (Swindells et al., J Mol Biol. 2017 Feb. 3; 429(3):356-364. doi: 10.1016/j.jmb.2016.08.019. Epub 2016 Aug 22 http://www.abysis.org/abysis/), the AbRSA tool (Li et al., Protein Sci. 2019 August; 28(8): 1524-1531, or the GenSmart™ Variable Domain Sequence Analyzer from Genscript.

In an embodiment, the capture agent is attached to a solid support. In an embodiment, the method further comprises coating the capture agent on the solid support. Such coating may be performed by contacting the solid support with a solution (e.g., a buffer) comprising a suitable amount of the capture agent. In an embodiment, the method further comprises at least one washing step after the coating step to remove the uncoated capture agent. The capture agent may be attached or coated on the solid support using a suitable tag, through its fragment crystallizable (Fc) domain (for antibodies), or through molecular interactions such as hydrophobic or hydrophilic intermolecular interactions.

The above-mentioned solid support may be any solid support which permits the binding (e.g., immobilization) of the capture agent and which may be used for the desired application. It includes for example glass or plastic plates/slides, beads/resins, etc. In an embodiment, the above-mentioned solid support is a plastic plate/slide. In embodiments, the above-mentioned plates/slides may be modified (e.g., coated, chemically modified, derivatized) prior to immobilization of the capture agent. In an embodiment, the solid support is modified to permit or facilitate the covalent or non-covalent immobilization of the capture agent, using any method known in the art. The solid support may be either amino- or carboxy-functionalized, depending on whether immobilization of the capture agent through its C- or N-terminal end is desired. The solid support may be modified/coated using any conventional moiety capable of binding to a corresponding moiety (affinity tag) conjugated to the capture agent, e.g., using typical affinity tags-based systems such as NTA-“His-Tag” systems, biotin—avidin/streptavidin systems, glutathione S-transferase (GST)—glutathione systems, Maltose Binding Protein (MBP)—amylose systems, as well as antigen—antibody systems.

In an embodiment, the detection agent comprises the first and second detection agents. In another embodiment, the detection agent comprises the third detection agent.

In an embodiment, the second, third, fourth and/or fifth sgp120-binding molecules is/are conjugated to a detectable label or reporter protein. In another embodiment, the second, third, fourth and/or fifth sgp120-binding molecules is/are not directly conjugated to a detectable label or reporter protein, and the detection agent further comprises a secondary agent that specifically binds to the second, third, fourth and/or fifth sgp120-binding molecules, such as an anti-human antibody conjugated to a detectable label or reporter protein.

The term “detectable label or reporter protein” as used herein refers to a moiety emitting a signal (e.g., light) that may be detected using an appropriate detection system. Any suitable detectable label may be used in the method described herein. Detectable labels and reporter proteins include, for example, enzyme or enzyme substrates, reactive groups, chromophores such as dyes or colored particles, luminescent moieties including bioluminescent, phosphorescent or chemiluminescent moieties, and fluorescent moieties. In an embodiment, the detectable label is a fluorescent moiety. Fluorophores that are commonly used include, but are not limited to, fluorescein, 5-carboxyfluorescein (FAM), 27-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl)aminonaphthalene-I-sulfonic acid (EDANS), Alexa™ (Molecular Probes), fluor dye, Bodipy dye™ (Life technologies), Cy dye™ (Life technologies), dansyl, umbelliferone (7-hydroxycoumarin), fluorescent microsphere, luminescent nanocrystal, Marina blue™ (Life technologies), Cascade blue™ (Life technologies), Cascade yellow™ (Life technologies), Pacific blue™ (Life technologies), Oregon green™ (Life technologies), Tetramethylrhodamine, Rhodamine, Texas red™ (Life technologies), as well as fluorescent proteins such as green fluorescent protein (GFP), cyan fluorescent protein (CFP), yellow fluorescent protein (YFP), and the like. Enzymes used for labeling proteins for immunoassays are known in the art, and include for example peroxidases (e.g., horseradish peroxidase, HRP) and alkaline phosphatase (AP). In an embodiment, the detectable label or reporter protein is an enzyme, preferably a peroxidase such as HRP.

The signal emitted by the detectable label or reporter protein (a signal that is proportional to the amount of detectable label or reporter protein present in the assay mixture) may be measured by any method known in the art. For example, if the detectable label or reporter protein is an enzyme, a substrate for the enzyme, preferably a chromogenic or fluorogenic substrate, is added to the assay mixture, and the signal is measured by detecting the level of a detectable product generated by catalysis of the substrate by the enzyme. For a chromogenic or fluorogenic substrate, the signal is measured by assessing the color or fluorescence intensity in the assay mixture using a suitable device, such as a spectrometer. Examples of chromogenic HRP substrates include 3,3′,5,5′-tetramethylbenzidine (TMB), 3,3′-Diaminobenzidine (DAB) and 2,2′-azino-di-[3-ethylbenzthiazoline-6-sulfonic acid](ABTS).

The method may include one or more washing steps. In an embodiment, the method comprises at least one washing step prior to detecting the presence of a second complex between the detection agent and the first complex, e.g., to remove the unbound conjugates. In another embodiment, the method comprises at least one washing step prior to adding the detection agent.

In an embodiment, the above-mentioned sample is a biological sample is a biological fluid, e.g., urine, saliva, lymph, cervicovaginal fluid, cerebrospinal fluid or a blood-derived sample. The term “blood-derived sample” as used herein refers to blood (e.g., fresh blood, stored blood) or to a fraction thereof, such as serum, plasma and the like. It also refers to any sample that may be obtained following one or more purification, enrichment, and/or treatment steps using blood (obtained by venous puncture, for example) as starting material. In an embodiment, the above-mentioned blood-derived sample is plasma.

In an embodiment, the present disclosure provides an immunoassay or method to detect or quantitate sgp120 in a sample, the method comprising:
coating a first solid surface with the capture agent defined herein;
contacting the coated first solid surface with the sample to form a complex between sgp120 in the sample (if present) and the capture agent;
removing unbound biological sample;
contacting the coated first solid surface with the detection agent defined herein to form a complex between the capture agent, the sgp120 in the sample (if present) and the detection agent;
washing the coated first solid surface;
contacting the coated first solid surface with a substrate capable of detecting the detectable marker (e.g., label, enzyme) presented in the detection agent; and
detecting or quantitating the detectable marker.

In an embodiment, the kit according to the present disclosure may be divided into separate packages or compartments containing the respective reagent components explained above.

In addition, such a kit may optionally comprise one or more of the following: (1) instructions for using the reagents for the detecting the presence or absence of sgp120 in a sample according to the methods described herein; (2) one or more containers; and/or (3) appropriate controls/standards. Such a kit can include reagents for collecting a biological sample from a patient and reagents for processing the biological sample. The kits featured herein can also include an instruction sheet describing how to perform the method for detecting the presence or absence of sgp120 in a sample.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing the method described herein and interpreting the results.

The presence of sgp120 in plasma from HIV-1-infected individuals is believed to be associated with immune dysfunctions related to residual immune dysregulation syndrome (RIDS) and cardiovascular diseases. Thus, the methods and kits described herein could be used to identify or stratify ART-treated HIV-1-infected individuals with RIDS based on their levels of sgp120 to determine whether these patients could benefit from treatment with agents capable of blocking the Env-CD4 interaction, e.g., HIV-1 attachment inhibitors such as temsavir and fostemsavir (see, e.g., Richard et al., 2023, Cell Chemical Biology 30, 540-552).

Thus, in another aspect, the present disclosure provides a method of identifying an HIV-infected individual suffering from or at risk of suffering from HIV-associated inflammaging or chronic immune activation (e.g., residual immune dysregulation syndrome (RIDS)), the method comprising performing the method/assay defined herein on a biological sample from the HIV-infected individual, wherein the presence of sgp120 in the sample is indicative that the individual suffers from or is at risk of suffering from HIV-associated inflammaging or chronic immune activation. In an embodiment, the method further comprises administrating a suitable therapy for treating the HIV-associated inflammaging or chronic immune activation in the individual, for example a therapy with an HIV-1 attachment inhibitor. Examples of HIV-1 attachment inhibitors include temsavir, fostemsavir and analogs thereof (see, e.g., Lai, Yen-Ting. “Small Molecule HIV-1 Attachment Inhibitors: Discovery, Mode of Action and Structural Basis of Inhibition.” Viruses vol. 13,5 843. 6 May. 2021, doi:10.3390/v13050843).

The present disclosure also provides a method of treating HIV-associated inflammaging or chronic immune activation (e.g., residual immune dysregulation syndrome (RIDS)) in an HIV-infected individual, the method comprising administering an effective amount of an HIV-1 attachment inhibitor to the HIV-infected individual suffering from HIV-associated inflammaging or chronic immune activation (e.g., RIDS) identified by the method described herein (based on the presence/level of sgp120 in a biological sample from the individual). The present disclosure also provides an HIV-1 attachment inhibitor for use in the treatment of HIV-associated inflammaging or chronic immune activation (e.g., RIDS) in an HIV-infected individual, wherein the HIV-infected individual suffering from HIV-associated inflammaging or RIDS is identified by the method described herein. The present disclosure also provides the use of an HIV-1 attachment inhibitor for the treatment of HIV-associated inflammaging or chronic immune activation (e.g., RIDS) in an HIV-infected individual, wherein the HIV-infected individual suffering from HIV-associated inflammaging or chronic immune activation (e.g., RIDS) is identified by the method described herein. The present disclosure also provides the use of an HIV-1 attachment inhibitor for the manufacture of a medicament for the treatment of HIV-associated inflammaging or chronic immune activation (e.g., RIDS) in an HIV-infected individual, wherein the HIV-infected individual suffering from HIV-associated inflammaging or chronic immune activation (e.g., RIDS) is identified by the method described herein.

In an embodiment, the HIV-1 attachment inhibitor is temsavir or fostemsavir.

### EXAMPLES

The present disclosure is illustrated in further details by the following non-limiting examples.

### Example 1: Materials and Methods

### Study Design

A cross-sectional study, nested within the CHACS [20], was designed. The specific objectives of the study were to quantify sgp120 in plasma of participants and study the relationship of sgp120 and anti-cluster A antibodies to correlates of immune dysfunction, inflammation, and subclinical CVD. Longitudinal samples from 9 sgp120-positive participants were also analyzed.

### Study Population

The CHACS (Canadian HIV and Aging Cohort Study) protocol has been described previously [20]. Briefly, it is a prospective cohort study and biobank, ongoing since 2012 in 10 clinical sites across Canada. Inclusion criteria are to be ≥40 years old or to have lived with HIV for ≥15 years and have a life expectancy of >1 year at enrollment. Participants with no known CVD and a 10-year Framingham risk score of overt CVD ranging from 5% to 20%, no allergy to contrast medium, and no renal failure, were invited to participate in the cardiovascular imaging substudy, in which they underwent computed tomography coronary angiography (CCTA), which was performed as previously described [21-23]. Participants were selected for the present study if they were recruited into the Montreal and Quebec CHAOS study sites and had undetectable viral loads. For all participants, data on sociodemographic characteristics, HIV disease history, and traditional cardiovascular risk factors are available through the CHAOS study database. Characteristics of Participants in the CHACS With Undetectable Human Immunodeficiency Virus Viremia, and participants to subgroups are depicted in Table 1.

TABLE 1
Characteristics of Participants in the CHACS With Undetectable Human
Immunodeficiency Virus Viremia, and participants to subgroups.

| | Study Participants, No. (%)^{a} | | | | | Multiplex | | |
|---|---|---|---|---|---|---|---|---|
| | | | Cardiovascular | | | Substudy | | |
| | Total Study | | Imaging | | | Participants, | | |
| | Population | | Substudy | | | No. (%)^{a} | | |
| Characteristic | (n = 386) | | (n = 145) | | P Value^{b} | (n = 157) | | P Value^{b} |
| Age, mean | 55.8 | (7.6) | 56.6 | (6.5) | .14 | 56.6 | (6.6) | .07 |
| (SD), years | | | | | | | | |
| Male sex | 347 | (90.0) | 134 | (92.4) | .23 | 144 | (91.7) | .39 |

Ethnicity

| | | | | | | | | |
|---|---|---|---|---|---|---|---|---|
| White | 317 | (82.1) | 123 | (84.8) | .39 | 131 | (83.4) | .29 |
| Black | 32 | (8.3) | 12 | (8.2) | | 15 | (9.6) | |
| Other | 37 | (9.6) | 10 | (6.9) | | 11 | (7.0) | |
| Duration of HIV | 18.3 | (12-25) | 18.3 | (12-25) | .27 | 19.0 | (14-25) | .52 |
| infection, | | | | | | | | |
| median (IQR), | | | | | | | | |
| years | | | | | | | | |
| Nadir CD4+ cell | 210 | (120-310) | 200 | (90-300) | .30 | 195 | (100-300) | .11 |
| count, median | | | | | | | | |
| (IQR), cells/mL | | | | | | | | |
| Nadir CD4+ cell | 155 | (49.1) | 67 | (52.8) | | 74 | (54.4%) | |
| count < 200/mL | | | | | | | | |
| Absolute CD4+ | 615 | (250) | 620 | (255) | .27 | 617 | (251) | .50 |
| cell count, | | | | | | | | |
| mean (SD), | | | | | | | | |
| cells/mL | | | | | | | | |
| Absolute CD8+ | 763 | (376) | 796 | (382) | .10 | 809 | (408) | .07 |
| cell count, | | | | | | | | |
| mean (SD), | | | | | | | | |
| cells | | | | | | | | |
| CD4/CD8 ratio, | 0.96 | (0.53) | 0.96 | (0.47) | .92 | 0.93 | (0.49) | .35 |
| mean (SD) | | | | | | | | |
| Duration of | 13.9 | (6.7) | 14.1 | (6.8) | .58 | 14.1 | (6.9) | .58 |
| ART, mean | | | | | | | | |
| (SD), years | | | | | | | | |

Smoking status

| | | | | | | | | |
|---|---|---|---|---|---|---|---|---|
| Never smoker | 128 | (33.5) | 42 | (29.4) | .22 | 48 | (31.0) | .44 |
| Past or | 254 | (66.5) | 101 | (70.6) | | 107 | (69.0) | |
| present smoker | | | | | | | | |
| Intravenous | 43 | (11.2) | 14 | (9.6) | .51 | 15 | (9.5) | .42 |
| drug use | | | | | | | | |
| Alcohol abuse | 31 | (8.0) | 11 | (7.6) | .84 | 11 | (7.0) | .57 |
| High blood | 113 | (29.7) | 45 | (31.5) | .56 | 51 | (32.7) | .31 |
| pressure | | | | | | | | |
| Diabetes | 37 | (9.6) | 15 | (10.4) | .71 | 18 | (11.5) | .30 |
| mellitus | | | | | | | | |
| Framingham | 9 | (5-16) | 9 | (6-13) | .72 | 9 | (6-13) | .58 |
| risk score, | | | | | | | | |
| median (IQR) | | | | | | | | |
| Log anti-cluster | 2.4 | (1.4) | 2.2 | (1.2) | .22 | 2.3 | (1.2) | .71 |
| A antibodies, | | | | | | | | |
| mean (SD), RU | | | | | | | | |

sgp120

| | | | | | | | | |
|---|---|---|---|---|---|---|---|---|
| Undetectable | 279 | (72.3) | 80 | (55.2) | <.001 | 83 | (52.9) | <.001 |
| Low | 68 | (17.6) | 40 | (27.6) | | 48 | (30.6) | |
| High | 39 | (10.1) | 25 | (17.2) | | 26 | (16.56) | |

Abbreviations: ART, antiretroviral therapy; gp120, glycoprotein 120; HIV, human immunodeficiency virus; IQR, interquartile range; RU, relative units; SD, standard deviation.
^{a}Data represent no. (%) of participants unless otherwise specified.
^{b}P values were obtained using Fisher exact test for categorical variables and k-sample equality of median test for continuous variables, and they represent comparisons between subcohort participants and nonparticipants.

### ELISA for Sqp120

Coating. The capture antibody solution (4 μg/ml) was produced by diluting the monoclonal antibody (mAb) C11 or bovine serum albumin (BSA) as negative control in Phosphate Buffered Saline (PBS) 1× from Wisent Inc. Then, 50 μl of the capture antibody solution was added per well in a 96-well white Maxisorp™ Fluonunc™ cert plate (Thermo Fischer Scientific). The plate was sealed using an adhesive polyester film for microplate (from VWR) and incubated at 4° C. for 16 h.

Blocking. Following incubation, plates were warmed to room temperature (RT) before removing the coating solution and adding 200 μl of blocking buffer. The blocking buffer is composed of 2% BSA in Washing buffer (0.1% Tween™-20 in Tris Buffered Saline (TBS) 1× (from Fischer Scientific)). The plate was then incubated with blocking buffer for 90 min at RT.

Sgp120 immobilization. After incubation, wells were washed 4 times with 200 μl of washing buffer. Following the washing steps, 50 μl of the standard and samples (heat inactivated diluted plasma 1:100 from HIV-infected or uninfected individuals) were added to the plate in triplicate. The standards were obtained by 2-fold serial dilution of monomeric soluble YU2 gp120 in immobilization buffer (one part of blocking buffer and 19 parts of washing buffer) (100 ng/ml to 195 μg/ml). The 2-fold serial dilution was performed with immobilization buffer containing HIV-1 negative plasma (1:100 dilution). Diluted monomeric gp120 were then spiked into HIV-1 negative plasma (1:100 dilution). The samples were also diluted with the immobilization solution (1:100 dilution). The plate was then incubated for 2 h at RT.

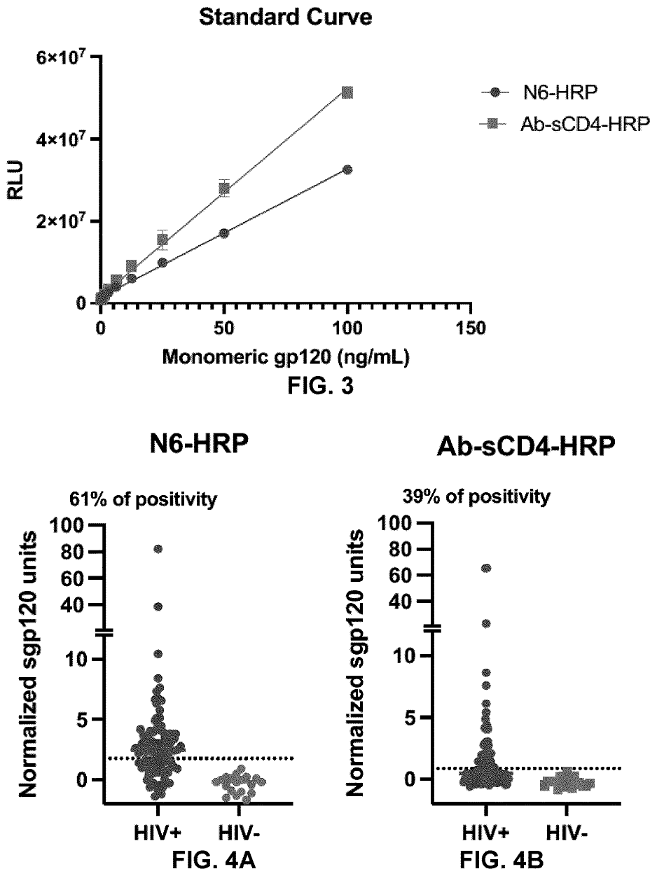

Detection. Following the incubation, the wells were washed 4 times with 200 μl of washing solution and then 50 μl of the detection antibody solution was added to each well. The detection antibody solution is composed of the N6 mAb conjugated to HRP diluted in the detection buffer (one part of blocking buffer and 5 parts of washing buffer) at 6 μg/ml. Alternatively, the detection antibody solution is composed of the combination of the 17b-sCD4 (3 μg/ml) and A32-sCD4 (3 μg/ml), both conjugated to HRP, diluted in the detection buffer. The plate was then incubated at RT for 90 min. Following incubation, the wells were washed 4 times with 200 μl of washing solution. HRP enzyme activity was then determined after the addition of a 1:1 mix of Western Lightning oxidizing and luminol reagents (Perkin Elmer Life Sciences, Waltham, MA, USA). Light emission was measured with a LB942 TriStar luminometer (Berthold Technologies). Signal obtained with BSA was subtracted for each plasma. A standard curve was established (FIG. 3) and used to normalize the signal obtained among different experiments. The positivity threshold was established using the following formula: mean of 20 HIV-negative plasma+(3 standard deviation of the mean of the 20 HIV-negative plasma). The positivity threshold for plasma with high levels of sgp120 was established using the following formula: mean of all 20 HIV-negative plasma+(6 standard deviation of the mean of all 20 HIV-negative plasma).

### Measurement of Anti-Cluster a Antibodies

For measurement of anti-cluster A antibodies, wells were coated with stabilized gp120 inner domain ID2 [26](0.1 μg/mL in PBS), in parallel with BSA (0.1 μg/mL in PBS). After blocking, the cluster A specific A32 mAb (1 μg/mL) or diluted plasma 1:1000 from HIV-infected or uninfected individuals were added to the well and detection of plasma antibodies was performed using HRP-conjugated goat-anti-human IgG (Invitrogen) at a dilution of 1:3000. Signal was measured as described above and signal obtained with BSA was subtracted for each plasma and were then normalized to the signal obtained with A32 mAb present in each plate.

### Measurement of CD4-Binding Site (CD4Bs) Antibodies

For measurement of CD4-binding site (CD4Bs) antibodies, wells were coated with resurfaced stabilized core 3 (RSC3) [47](0.1 μg/mL in PBS). In parallel, wells were coated with BSA (0.1 μg/mL in PBS). After blocking, 2G12 mAb (1 μg/mL), VRC01 mAb (1 μg/mL) or diluted plasma 1:1000 from HIV-infected or uninfected individuals were added to the well and detection of plasma antibodies was performed using HRP-conjugated goat-anti-human IgG (Invitrogen) at a dilution of 1:3000. Signal was measured as described above and signal obtained with BSA was subtracted for each plasma and were then normalized to the signal obtained with 2G12 mAb present in each plate.

### Protein Production and Purification

Production and purification of monomeric soluble HIV-1_{YU2} gp120 was described elsewhere [31, 24]. Briefly, recombinant HIV-1_{YU2} gp120 was produced using a plasmid (pcDNA3.1) encoding the codon-optimized full-length HIV-1_{YU2}gp120 containing a C-terminal hexa-histidine tag [46]. FreeStyle™ 293F cells (Thermo Fisher Scientific) were grown in FreeStyle™ 293F medium (Thermo Fisher Scientific) to a density of 1×10⁶ cells/mL at 37° C. with 8% CO₂ with regular agitation (150 rpm). Cells were transfected with the gp120 expressor using ExpiFectamine™ 293 transfection reagent, as directed by the manufacturer (Thermo Fisher Scientific). One week later, cells were pelleted, and supernatants were filtered using a 0.22-μm-pore-size filter (Thermo Fisher Scientific). Recombinant gp120 was purified by nickel affinity columns, as directed by the manufacturer (Thermo Fisher Scientific). Monomeric gp120 was subsequently purified by fast protein liquid chromatography (FPLC), as previously reported [24]. The purification by FPLC was performed using an ÅKTAprime™ Plus FPLC with a HiLoad™ 16/60 Superdex™ 200 PG (GE Healthcare, Chicago, IL, USA). The gp120 preparations were dialyzed against phosphate-buffered saline (PBS) and stored in aliquots at −80° C. until further use. To assess purity, recombinant proteins were loaded on non-reducing SDS-PAGE polyacrylamide gels and stained with Coomassie blue.

### Multiplex Measurements of Soluble Inflammatory Markers.

Duplicates of HIV-1 inactivated plasma samples were analyzed using a customized Human Magnetic Luminex™ Assay (LXSAHM-14, LXSAHM-1 or LXSAHM-2, R&D Systems). Plates were read using a MAGPIX™ system (Luminex) and analyzed with the software xPONENT™ v.4.3.229.0. Plasma samples presenting markers below the limit of detection were adjusted to 0. Results were adjusted according to sample dilution to represent the concentration in pg/mL of plasma.

### Measurement of HIV DNA.

Briefly, CD4⁺ T cells were isolated from PBMCs by negative selection using the EasySep™ Human CD4⁺ T cell Enrichment Kit (StemCell). HIV DNA was co-extracted using the AIIPrep™ DNA/RNA Mini Kit (Qiagen). HIV DNA (LTR-gag) copies were measured using ultrasensitive nested PCRs [48]. Results were expressed as HIV DNA copies per million CD4⁺ T cells.

### Statistical Analysis.

Differences in baseline characteristics between the participants included into subgroups and the remainder of the samples were assessed using Fisher's exact and k-median tests for categorial and continuous variables, respectively. We hypothesized a priori that anti-cluster A antibodies and levels of sgp120 would interact to cause immune dysfunction and organ damage. Therefore, the associations between anti-cluster A antibodies and CD4 levels, CD4:CD8 ratios were modelled using linear regression models, including levels of sgp120 modelled as categorical (undetectable, low or high) and an interaction term between anti-cluster A antibodies and sgp120 levels. The association between the levels of sgp120, anti-cluster A antibodies and cardiovascular disease was modelled using logistic regression for the outcome of presence vs absence of coronary artery plaque (TPV=0 v. TPV>0). In participants with subclinical cardiovascular disease (TPV>0) and detectable levels of sgp120, logistic regression was performed for association between the levels of sgp120, anti-cluster A antibodies and the combination of sgp120 and anti-cluster A antibodies with the total volume of coronary atherosclerotic plaque. All models were adjusted for potential confounders (identified a priori based on clinical knowledge) using a parsimonious approach: potential confounders were kept into the models if they modified the point estimate for the main association by more than 10%. Absence of multicollinearity was ensured by examination of correlation matrices. Missing data for covariates was simulated by multiple imputations by chained equations. Correlations between multiplex biomarkers were calculated using Spearman's Rank test and graphed as circular network in undirected mode using ggraph, igraph, and RColorBrewer packages in program R (R Core Team, 2014). Statistical analysis was conducted using Graphpad™ Prism version 8.4.2, Stata version 17 and R. For all analysis, alpha was set to 0.05. All hypothesis tests were two-sided, and no corrections were made for multiple testing.

### Example 2: Development of the ELISA for Sgp120 Plasma Detection

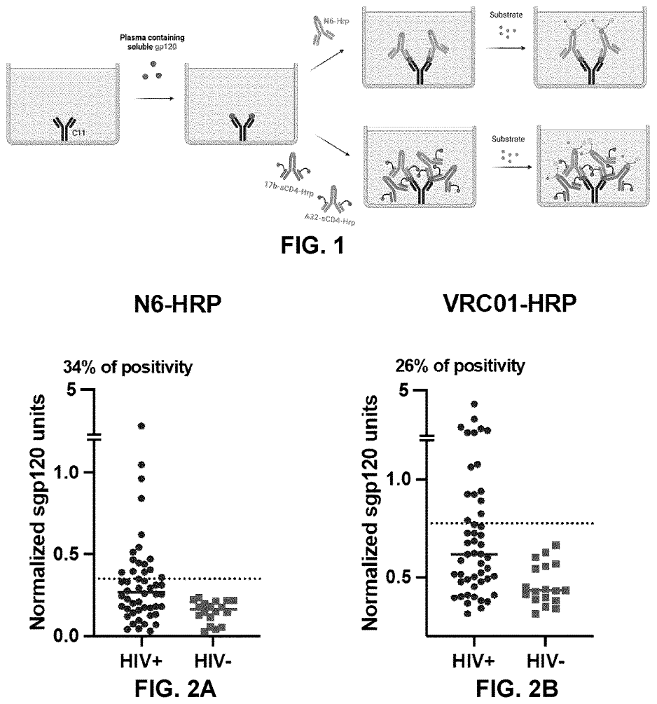

A schematic of an assay according to an embodiment of the disclosure is depicted in FIG. 1. ELISA plates were coated with the C11 Ab, a gp120 inner-domain-specific antibody that targets the highly conserved N-termini and 8-stranded β-sandwich structure of gp120 formed at the late stage of HIV-1 entry, and then incubated with a biological sample suspected of containing sgp120 such as diluted plasma from an HIV-infected subject. The C11 Ab was used as a bait (capture agent) to grab the sgp120 present in the plasma. The epitope recognized by the C11 Ab is buried on the trimeric Env present on virions or infected cells but exposed on sgp120.

Following washing steps, the detection agent (N6-HRP or the combination of 17b-sCD4-HRP/A32-sCD4-HRP) was added to the plate to attach to sgp120. N6 is a broadly neutralizing CD4 binding site (CD4BS) antibody that does not compete for C11 binding, and which was reported to target up to 98% of global HIV-1 isolates [28].

Following washing steps, an HRP substrate solution was added to initiate the reaction and generate a light signal if the detection agent was bound to sgp120 captured by the C11 Ab on the plate. Light emission was measured with a luminometer.

FIGS. 2A and 2B show the detection of soluble gp120 in plasma from HIV-1-infected individuals using the capture antibody C11 in combination with N6-HRP (FIG. 2A) or the classical CD4-binding site neutralizing Ab VRC01-HRP (FIG. 2B). A higher proportion HIV+ plasma was identified as sgp120+ using the N6 mAb (34%) relative to VRC01 (26%), suggesting that the N6 antibody is superior to VRC01 at detecting immobilized sgp120.

The results in FIG. 4B show that a combination of two antibody-CD4 (Ab-CD4) hybrid molecules conjugated with HRP also permits to reveal the presence of sgp120 in plasma samples from chronically-infected ART-treated subjects (with undetectable viral loads). These single chain Ab-CD4 consist of a coreceptor binding site (CoRBS) Abs (17b) or a cluster A-specific Abs (A32) linked to the C terminus of soluble CD4 (sCD4). The 17b Abs recognize a conserved epitope within the bridging sheet of the CoRBS, while A32 targets an epitope that map to the highly conserved constant region 1 and 2 (C1-C2) of the gp120 inner domain. These epitopes are poorly exposed on the trimeric Env present on infected cells or HIV-1 particle but exposed on sgp120. The advantage of using this combination of Ab-sCD4 is the possibility to simultaneously target three highly conserved domains of gp120 that do not overlap with the C11 epitope: the CD4bs (via the sCD4 subpart), the CoRBS (via the 17b Ab subpart) and the inner domain (via the A32 Ab subpart). The CD4bs and the CoRBS of HIV-1 Env are important for viral entry and therefore highly conserved among HIV-1 isolate. The cluster A epitope is also highly conserved as it maps the interior of HIV-1 Env trimer at the gp41-gp120 interface that is directly involved in interprotomer contact that stabilizes the trimer. Accordingly, these two Ab-CD4 were shown to target HIV-1 Env from multiple HIV-1 clades.

Thus, the assay described herein permits to specifically detect and measure sgp120 in plasma from chronically-infected antiretroviral therapy (ART)-treated (with undetectable viral loads) individuals relative to heathy HIV uninfected donors. These ELISA assays could therefore be used to measure sgp120 in biological fluids and to evaluate the impact of sgp120 in immune dysfunction in ART-treated HIV-1-infected individuals. Preliminary results generated with the C11-N6 ELISA suggest that sgp120 could be associated with immune dysfunctions related to RIDS and cardiovascular diseases. Chronically-infected ART-treated individuals were stratified relative to sgp120 levels detected with the assay described herein (sgp120 negative, low and high). Interestingly, a negative correlation between CD4 counts and the levels of CD4-induced “toxic” ADCC-mediating Abs, reported to eliminate uninfected bystander CD4+ T cells coated with sgp120 (Richard et al., 2018; Richard et al., 2016b), was observed. Of note, this became significant in individuals presenting high levels of sgp120. These results suggest that the level of sgp120 and toxic ADCC-mediating Abs could contribute to prevent the restoration of circulating CD4+ T cell in ART-treated individuals. Moreover, a significant correlation between the combined presence of sgp120 and CD4-induced ADCC-mediating Abs (designed as toxicity score) and subclinical coronary artery disease (defined by the presence of atherosclerotic plaque on cardiac computed tomography angiography) was also observed among this population.

Temsavir (BMS-626529, GSK2616713) is a novel attachment inhibitor that can prevent Env-CD4 interaction (Meanwell et al., 2018; Pancera et al., 2017). Temsavir is the active metabolite of fostemsavir (BMS-663068; GSK3684934, Rukobia), a molecule recently approved in the United States, Europe and Canada in combination with other antiretroviral treatments (ARVs) for adults with multidrug-resistant HIV-1 who are otherwise unable to construct a suppressive ARV regimen due to resistance, prior intolerance, or safety concerns. It was also recently demonstrated that temsavir can also block the immunoregulatory activities of sg120 in vitro (Richard et al., 2023). This molecule was found to prevent gp120 shed from infected cells from interacting with CD4 present in uninfected immune cells, thus protecting uninfected bystander CD4+ T cells from ADCC responses and inhibiting gp120-induced cytokine burst. However, whether fostemsavir could provide similar benefits in vivo in patients with RIDS by restoring circulating CD4+ T cells and/or reducing immune activation/inflammation, remain to be determined (see, e.g., Richard et al., 2023, Cell Chemical Biology 30, 540-552). The assay described herein could be used to stratify or identify ART-treated HIV-1-infected individuals with RIDS based on their levels of sgp120 to determine whether fostemsavir could have additional clinical benefits in these patients.

### Example 3: Measuring Sgp120 in Plasma from PLWH with Undetectable Viremia

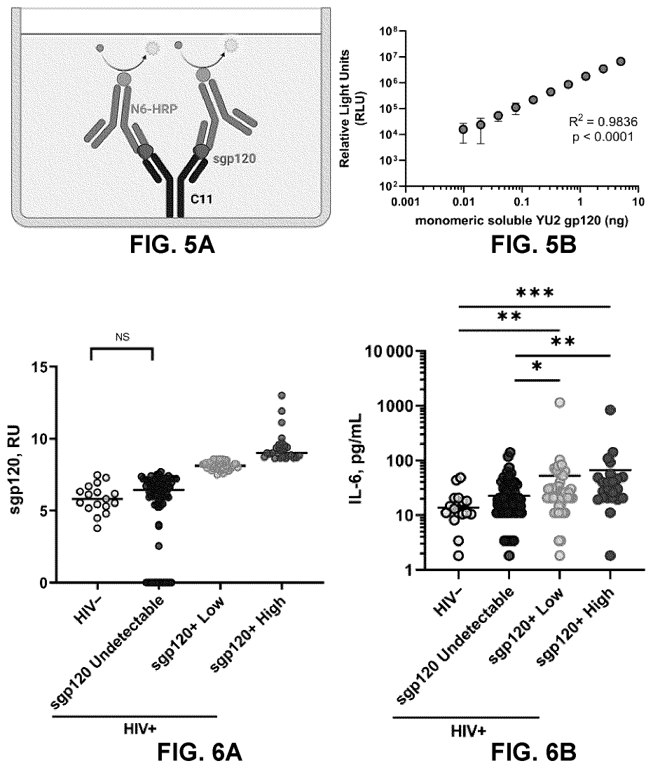

Sgp120 in plasma from PLWH With Undetectable Viremia was detected using the optimized assay depicted in FIG. 5A using the C11 antibody as the capture agent to grab the sgp120 present in the plasma, and the N6 antibody as a detection agent. Using this assay, a strong linearity (r=0.9836; P<0.001) over a 500-fold range was observed between the signal obtained and the quantity of purified recombinant monomeric soluble HIV-1YU2 gp120 used. This assay enabled the measurement of sgp120 in plasma samples from PLWH with undetectable viral loads compared with uninfected individuals (FIG. 6A). PLWH were further stratified into 3 subgroups based on the positivity threshold established with the uninfected plasma samples: (1) undetectable sgp120, (2) low levels of sgp120, and (3) high levels of sgp120 (FIG. 6A). Of the 386 plasma samples analyzed, 72.3% (n=279) had undetectable sgp120, 17.6% (n=68) had low levels, and 10.1% (n=39) had high levels (see Table 1).

### Example 4: Association of Anti-Cluster a Antibody Levels with Correlates of Immune Dysfunction

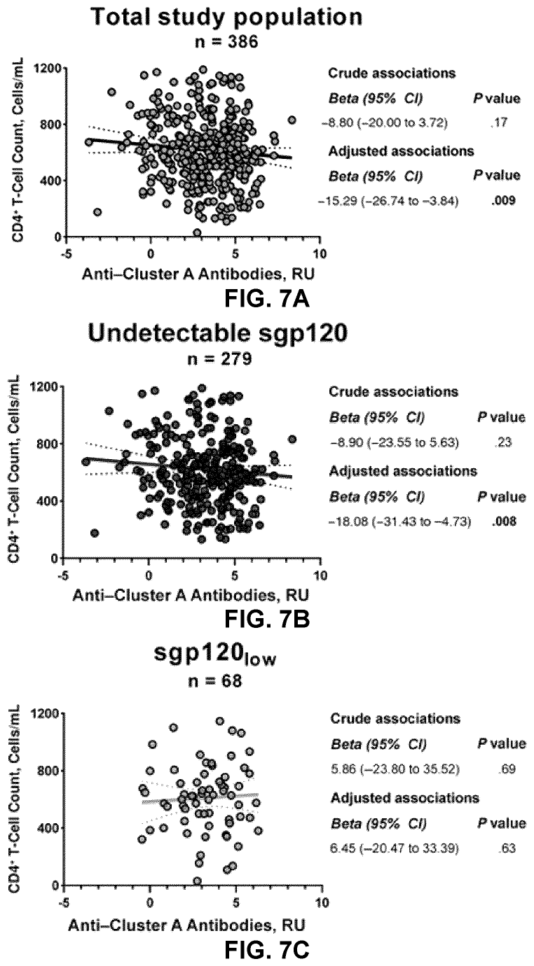

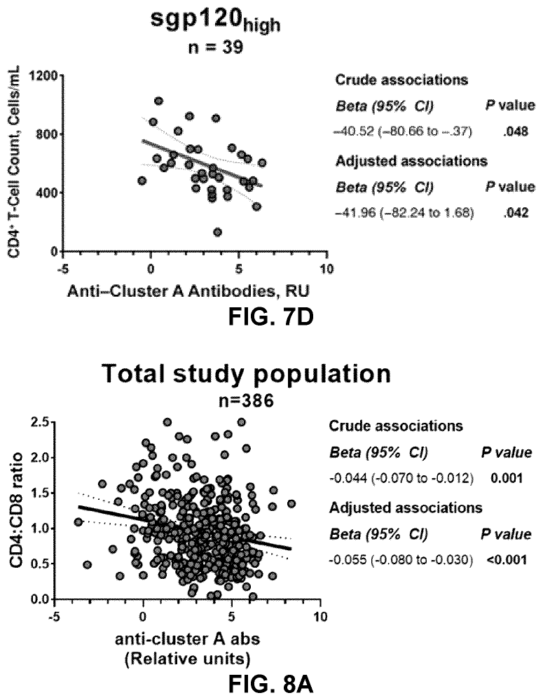

It has been previously reported that the release of sgp120 from infected cells sensitizes uninfected bystander CD4⁺ T cells to ADCC mediated by HIV-positive plasma in vitro, but whether this happens in PLWH remains unclear [15-17]. The levels of anti-cluster A antibodies were measured using an engineered stabilized gp120 inner-domain protein (ID2) exposing only the cluster A region [29, 30]. FIGS. 7A-D present the associations between anti-cluster A antibodies and absolute CD4⁺ cell counts or the CD4/CD8 ratio. In the total study population, after adjustment for potential confounders (including age, sex, ethnicity, smoking status, nadir CD4⁺ cell count, duration of ART, and levels of anti-CD4BS antibodies), each 1-log₂ increase in anti-cluster A antibody levels was associated with a mean predicted decrease in CD4⁺ cell count of −15.3×10⁶/mL (95% confidence interval [CI], −26.7×10⁶/mL to −3.8×10⁶/mL; P=0.009) (FIG. 7A).

TABLE 2
Association between the levels of anti-cluster A antibodies and absolute CD4 count or
CD4:CD8 ratio, stratified by levels of soluble gp120 in 386 PLWH with undetectable HIV viremia

| | | | Adjusted associations^{b} | | |
|---|---|---|---|---|---|
| Crude associations | | | | | Interaction |
| N | Beta [95% CI]^{a} | P value | Beta [95% CI] | P value | p-value |

CD4 count

| | | | | | | | | |
|---|---|---|---|---|---|---|---|---|
| Total study | 386 | −8.8 | (−20.00 to 3.72) | 0.17 | −15.29 | (−26.74 to −3.84) | 0.009 | NA |
| population | | | | | | | | |
| sgp120 | 279 | −8.90 | (−23.44 to 5.63) | 0.23 | −18.08 | (−31.43 to −4.73) | 0.008 | 0.11 |
| undetectable | | | | | | | | |
| Low levels of | 68 | 5.86 | (−23.80 to 35.52) | 0.69 | 6.45 | (−20.47 to 33.39) | 0.63 | |
| sgp120 | | | | | | | | |
| High levels of | 39 | −40.52 | (−80.66 to −0.37) | 0.048 | −41.96 | (−82.24 to −1.68) | 0.042 | |
| sgp120 | | | | | | | | |

CD4:CD8 ratio

| | | | | | | | | |
|---|---|---|---|---|---|---|---|---|
| Total study | 386 | −0.044 | (−0.070 to −0.012) | 0.001 | −0.055 | (−0.080 to −0.030) | <0.001 | NA |
| population | | | | | | | | |
| sgp120 | 279 | −0.039 | (−0.068 to 0.009) | 0.011 | −0.053 | (−0.081 to −0.024) | <0.001 | 0.21 |
| undetectable | | | | | | | | |
| Low levels of | 68 | −0.036 | (−0.103 to 0.032) | 0.30 | −0.035 | (−0.099 to −0.030) | 0.29 | |
| sgp120 | | | | | | | | |
| High levels of | 39 | −0.126 | (−0.211 to −0.041) | 0.005 | −0.130 | (−0.215 to −0.044) | 0.004 | |
| sgp120 | | | | | | | | |

^{a}Beta represents the mean predicted change in absolute CD4 cell counts or CD4:CD8 ratio for each 1log₂ increase in titers of anti-cluster A abs
^{b}Models are adjusted for age, sex, ethnicity, smoking, duration of antiretroviral therapy, nadir CD4 cell counts and levels of anti-CD4 binding sites, using a parsimonious model building strategy, where potential confounders, identified based on a priory clinical knowledge, were kept into the final model if they modified the point estimate of the main association in a bivariate model by 10% or more. Absence of multicollinearity was ensured by examination of correlation matrices.
Abbreviations:
HIV, human immunodeficiency virus;
PLWH, people living with HIV;
gp120, glycoprotein 120;
CI, confidence interval;
NA, not applicable

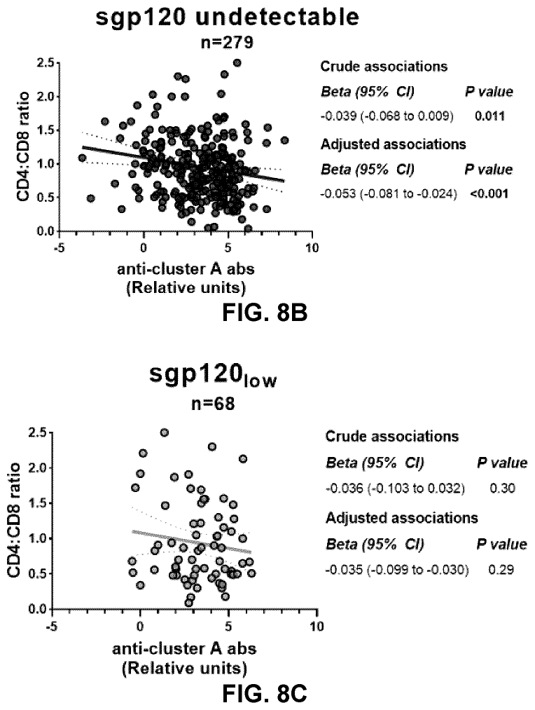

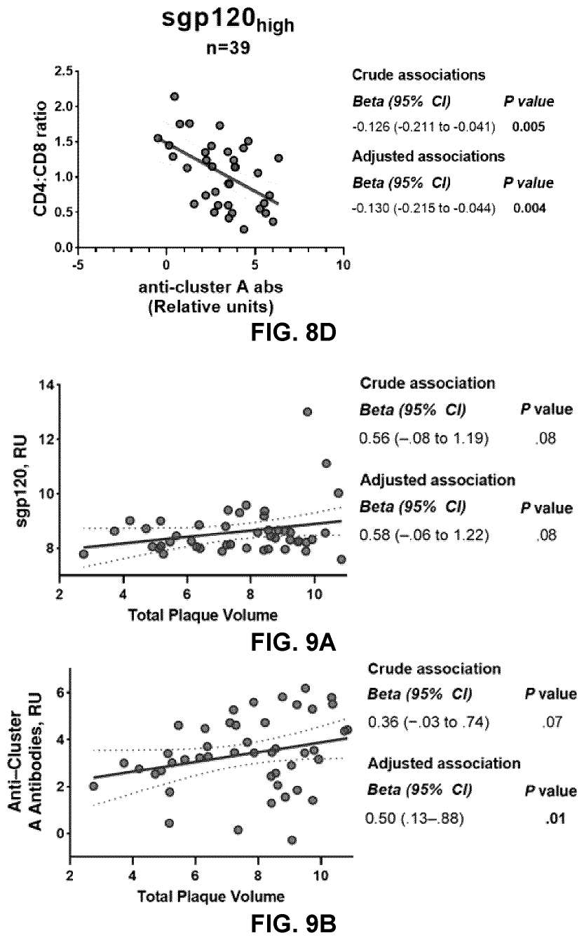

On stratification of the 386 PLWH by sgp120 levels, it was observed that the mean predicted change in CD4⁺ cell counts varies by stratum of sgp120. For those with sgp120 below detection levels, each increase of 1-log₂ in anti-cluster A antibodies is associated with a mean predicted decrease in absolute CD4⁺ cell count of −18.1×10⁶/mL (P=0.008) (FIG. 7B), while the mean predicted decline for participants with high levels of sgp120 is −42.0×10⁶/mL (P=0.04) (FIG. 7D). However, the P value for the interaction was 0.11 (Table 2). A similar dynamic is observed with the CD4/CD8 ratio (presented in FIGS. 8A-D and Table 2); within the total study population, each 1-log₂ increase in the levels of anti-cluster A antibodies is associated with a mean projected decrease in the CD4/CD8 ratio of −0.06 (95% CI, −0.08 to −0.03; P<0.001) (FIG. 8A).

The magnitude of this association was more pronounced in the subgroup with high levels of sgp120, in which the mean predicted decline in CD4/CD8 ratio was −0.13 (95% CI, −0.22 to −0.04; P=0.004) (FIG. 8D). However, this difference between the groups was not statistically significant (the P value for interaction between sgp120 levels and anti-cluster A antibodies was 0.21) (Table 2). Of note, no association was observed between anti-cluster A antibodies and CD4⁺ T-cell count or CD4/CD8 ratio in individuals presenting with low levels of sgp120 (FIG. 7C, FIG. 8C, and Table 2). Antibodies targeting the CD4BS were also measured, using the gp120 resurfaced stabilized core 3, a previously described probe exposing the CD4BS [31]. It was found that only 66 of 386 individuals (17%) had detectable levels of CD4BS antibodies; from which 44 had undetectable sgp120 levels, 13 had low sgp120 levels, and 9 had high sgp120 levels. No associations between CD4BS antibody levels and CD4⁺ T-cell counts were observed.

### Example 5: Association Between Sgp120 and Proinflammatory Markers

The HIV-1 gp120 is a pleiotropic molecule beyond its key role in viral entry [4, 7, 14, 15]. The presence of sgp120 was associated with increased levels of proinflammatory markers in the plasma of early and acute PLWH [7]. To test whether this observation could be extended to PLWH receiving long-term ART treatment, multiplex measurements of various soluble markers associated with chronic inflammation were performed in a subset of 157 PLWH. The results show that plasma levels of IL-6 are significantly higher in people with low or high levels of sgp120 than in the sgp120-undetectable group and uninfected controls (FIG. 6B). Interestingly, a positive correlation was observed between sgp120 and TNF-α levels in the group with high sgp120 levels (Table 3); r=0.410, P=0.04). The correlations between all inflammatory biomarkers measured by multiplex platform, as a function of detectable levels of sgp120. While the undetectable group has weak associations among the parameters measured, the presence of low or high levels of sgp120 intensifies the network of associations. Notably, the inverse association between soluble CD14 with lymphocyte counts and CD4/CD8 ratios is strengthened in the presence of sgp120. These results suggest that sgp120 acts as an effect modifier, modulating the associations among the different biomarkers analyzed in the present study.

TABLE 3
Associations* between sgp120 and anti-cluster A antibodies with clinical
and laboratory markers in 157 PLWH with multiplex measurements

| | | |
|---|---|---|
| sgp120 undetectable | sgp120_{low} | sgp120_{high} |

Anti-cluster A antibodies

| | | | |
|---|---|---|---|
| % CD8 | S_{Rho} = 0.311; p = 0.004 | n.s | S_{Rho} = 0.447; p = 0.022 |
| CD8 count | S_{Rho} = 0.256; p = 0.021 | n.s | n.s |
| CD4:CD8 | S_{Rho} = −0.272; p = 0.013 | n.s | S_{Rho} = −0.449; p = 0.021 |
| ratio | | | |
| CD4 count | n.s | n.s | S_{Rho} = −0.396; p = 0.045 |
| Total HIV | n.s | n.s | S_{Rho} = 0.577; p = 0.008 |
| DNA | | | |

soluble gp120

| | | | |
|---|---|---|---|
| High density | n.s | n.s | S_{Rho} = −0.436; p = 0.026 |
| lipoproteins | | | |
| TNF-α | n.s | n.s | S_{Rho} = 0.410; p = 0.038 |
| sCD163 | n.s | n.s | S_{Rho} = −0.510; p = 0.008 |

*Spearman Rank correlations.
Abbreviations:
n.s, not significant;
sgp120, soluble gp120

### Example 6: Association of Sgp120 and Anti-Cluster a Antibodies with the Size of Coronary Artery Plaque in Participants with Subclinical CVD and Detectable Sgp120

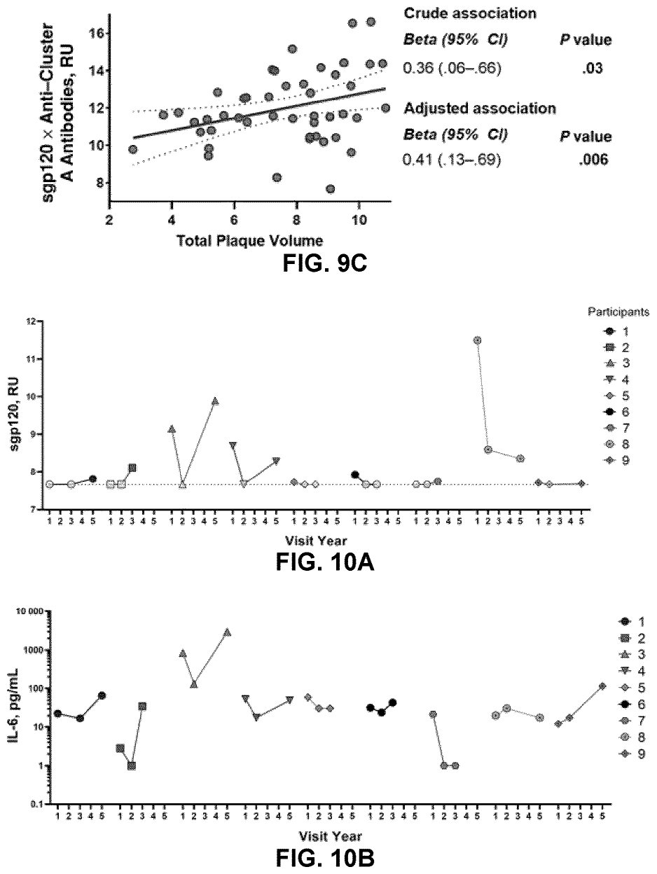

In the subgroup of 145 participants with available cardiovascular imaging, 97 (67%) presented with ≥1 coronary artery plaque measurable on CCTA, defining subclinical CVD. Levels of sgp120 (adjusted odds ratio, 1.04 [95% CI, 0.96-1.12]), anti-cluster A antibodies (1.01 [0.82-1.24), or a score defined by multiplying the levels of sgp120 and anti-cluster A antibodies (1.03 [0.96-1.1]) were not associated with the presence or absence of CVD. However, among the 46 individuals with detectable subclinical CVD and detectable sgp120, an association between the size of the coronary artery plaques and anti-cluster A antibodies was found (P=0.01), as well as of the multiplicative score of anti-cluster A antibodies with levels of sgp120 (P=0.006) (FIGS. 9A-C) and Table 4), after adjusting for potential confounders (age, sex, hypertension, diabetes mellitus, lipid levels, smoking status, alcohol use disorder, intravenous drug use, and duration of ART). These analyses were repeated using the low attenuation fraction of coronary artery plaque volume (a marker of high-risk plaque) as opposed to the total volume of coronary artery plaque, and the associations were unchanged (Table 4).

TABLE 4
Association between anti-cluster A antibodies, sgp120 levels and
their combination with the size of coronary artery plaque in 46
participants with both cardiovascular disease and detectable sgp120

| | Crude association | | Adjusted associations* | |
|---|---|---|---|---|
| | Beta [95% CI] | P value | Beta [95% CI] | P value |
| Total coronary plaque volume | | | | |
| Log₂ anti-cluster A | 0.36 (−0.03 to 0.74) | 0.067 | 0.50 (0.13 to 0.88) | 0.010 |
| antibodies levels | | | | |
| Log₂ sgp120 levels | 0.56 (−0.08 to 1.19) | 0.083 | 0.58 (−0.06 to 1.22) | 0.076 |
| Combination of Log | 0.36 (0.06 to 0.66) | 0.03 | 0.41 (0.13 to 0.69) | 0.006 |
| anti-cluster A antibodies | | | | |
| and log sgp120 levels | | | | |

Total low attenuation plaque volume

| | | | | |
|---|---|---|---|---|
| Log₂ anti-cluster A | 0.36 (−0.06 to 0.79) | 0.094 | 0.52 (0.11 to 0.95) | 0.014 |
| antibodies levels | | | | |
| Log₂ sgp120 levels | 0.54 (−0.17 to 1.26) | 0.13 | 0.56 (−0.16 to 2.75) | 0.12 |
| Combination of Log | 0.36 (0.02 to 0.70) | 0.036 | 0.41 (0.10 to 0.73) | 0.012 |
| anti-cluster A antibodies | | | | |
| and log sgp120 levels | | | | |

*Models are adjusted for age, sex, smoking, low/high density lipoproteins, diabetes, hypertension, intravenous drug use, alcohol use disorder and duration of antiretroviral therapy, using a parsimonious model building strategy, where potential confounders, identified based on a priory clinical knowledge, were kept into the final model if they modified the point estimate of the main association in a bivariate model by 10% or more.
Total low attenuation plaque volume is defined as total volume with density of less than 30 hounsfield units.
Abbreviations: gp120, glycoprotein 120; CI, confidence interval.

### Example 7: Longitudinal Analysis of Sgp120

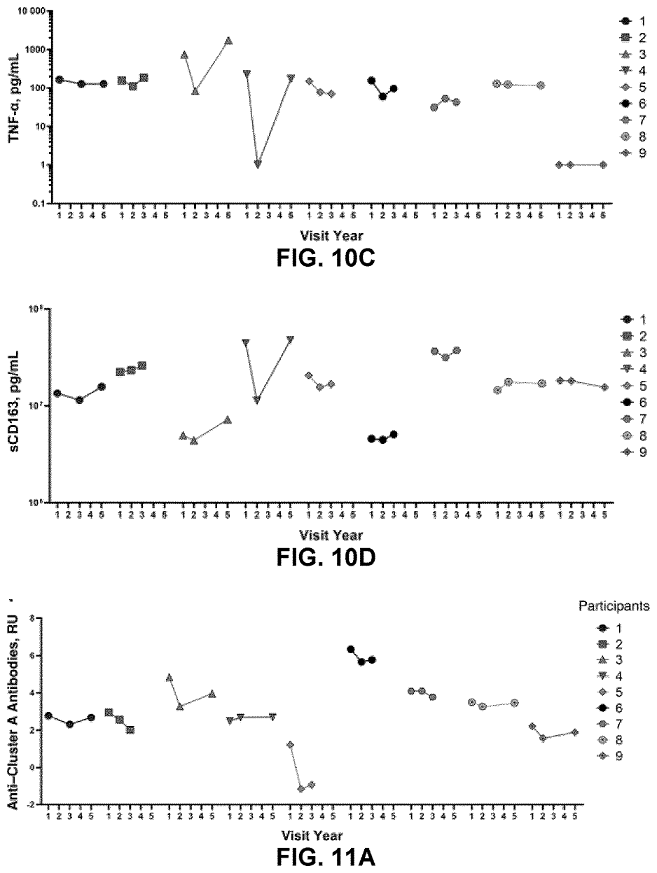

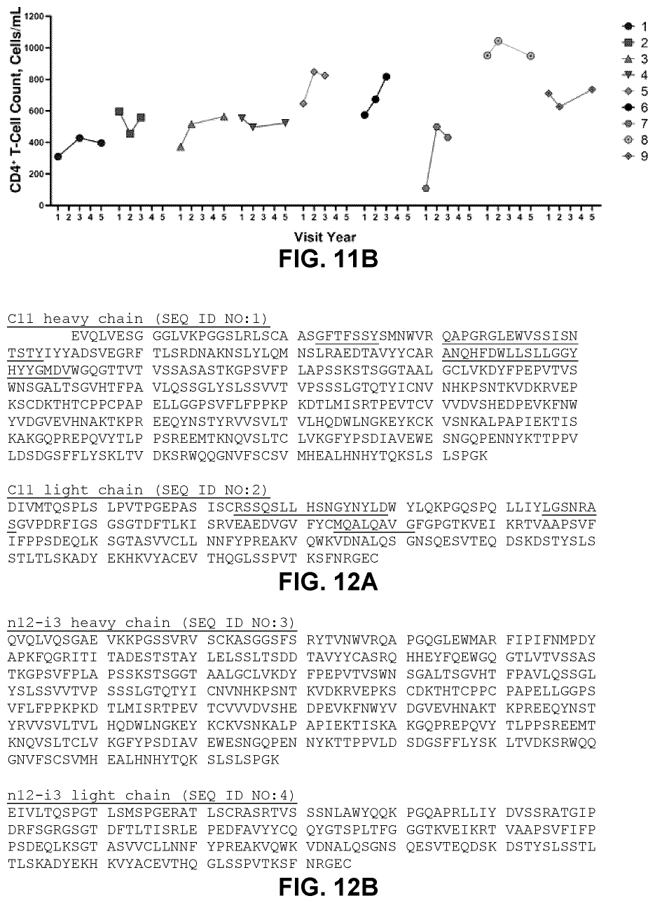

Plasma samples from 9 sgp120-positive PLWH in the CHACS cohort with no viremia were analyzed. These individuals had 3 sample visits ranging from 1 to 5 years after the baseline assessment. sgp120, anti-cluster A antibodies, and 3 inflammatory markers (IL-6, TNF-α, and soluble CD163 [sCD163]) were assessed. It was found that sgp120 levels are dynamic (FIG. 10A). Interestingly, IL-6, TNF-α and sCD163 followed a similar pattern to that of sgp120 in most participants (FIGS. 10B-D). While this does not prove causality, it suggests an association among these markers and sgp120. Further supporting the hypothesis that anti-cluster A antibodies are negatively associated with CD4⁺ T-cell counts, opposite trends were observed between anti-cluster A Abs and CD4⁺ T-cell counts in 6 of these 9 participants (FIGS. 11A-B).

Although the present invention has been described herein above by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Marcus J L, Leyden W A, Alexeeff S E, et al. Comparison of overall and comorbidity-free life expectancy between insured adults with and without HIV infection, 2000-2016. JAMA Netw Open 2020; 3:e207954.
2. Rodes B, Cadinanos J, Esteban-Cantos A, Rodriguez-Centeno J, Arribas JR. Ageing with HIV: challenges and biomarkers. eBioMedicine 2022; 77:103896.
3. Lederman M M, Funderburg N T, Sekaly R P, Klatt N R, Hunt P W. Residual immune dysregulation syndrome in treated HIV infection. Adv Immunol 2013; 119:51-83.
4. Chirmule N, Pahwa S. Envelope glycoproteins of human immunodeficiency virus type 1: profound influences on immune functions. Microbiol Rev 1996; 60:386-406.
5. Del Corno M, Donninelli G, Varano B, Da Sacco L, Masotti A, Gessani S. HIV-1 gp120 activates the STAT3/interleukin-6 axis in primary human monocyte-derived dendritic cells. J Virol 2014; 88:11045-55.
6. Nazli A, Kafka J K, Ferreira V H, et al. HIV-1 gp120 induces TLR2- and TLR4-mediated innate immune activation in human female genital epithelium. J Immunol 2013; 191:4246-58.
7. Rychert J, Strick D, Bazner S, Robinson J, Rosenberg E. Detection of HIV gp120 in plasma during early HIV infection is associated with increased proinflammatory and immunoregulatory cytokines. AIDS Res Hum Retroviruses 2010; 26:1139-45.
8. Helseth E, Olshevsky U, Furman C, Sodroski J. Human immunodeficiency virus type 1 gp120 envelope glycoprotein regions important for association with the gp41 transmembrane glycoprotein. J Virol 1991; 65:2119-23.
9. Yang X, Mahony E, Holm G H, Kassa A, Sodroski J. Role of the gp120 inner domain beta-sandwich in the interaction between the human immunodeficiency virus envelope glycoprotein subunits. Virology 2003; 313:117-2.
10. Finzi A, Xiang S H, Pacheco B, et al. Topological layers in the HIV-1 gp120 inner domain regulate gp41 interaction and CD4-triggered conformational transitions. Mol Cell 2010; 37:656-67.
11. Santosuosso M, Righi E, Lindstrom V, Leblanc P R, Poznansky M C. HIV-1 envelope protein gp120 is present at high concentrations in secondary lymphoid organs of individuals with chronic HIV-1 infection. J Infect Dis 2009; 200:1050-3.
12. Oh S K, Cruikshank W W, Raina J, et al. Identification of HIV-1 envelope glycoprotein in the serum of AIDS and ARC patients. J Acquir Immune Defic Syndr 1992; 5:251-6.
13. Herbein G, Khan K A. Is HIV infection a TNF receptor signalling-driven disease? Trends Immunol 2008; 29:61-7.
14. Levast B, Barblu L, Coutu M, et al. HIV-1 gp120 envelope glycoprotein determinants for cytokine burst in human monocytes. PLoS One 2017; 12:e0174550.
15. Richard J, Prévost J, Bourassa C, et al. Temsavir blocks the immunomodulatory activities of HIV-1 soluble gp120. Cell Chem Biol 2023; 30:540-52.e6.
16. Richard J, Veillette M, Ding S, et al. Small CD4 mimetics prevent HIV-1 uninfected bystander CD4+ T cell killing mediated by antibody-dependent cell-mediated cytotoxicity. EBioMedicine 2016; 3:122-3.
17. Richard J, Prevost J, Baxter A E, et al. Uninfected bystander cells impact the measurement of HIV-specific antibody-dependent cellular cytotoxicity responses. mBio 2018; 9:e00358-18.
18. Bonsignori M, Pollara J, Moody M A, et al. Antibody-dependent cellular cytotoxicity-mediating antibodies from an HIV-1 vaccine efficacy trial target multiple epitopes and preferentially use the VH1 gene family. J Virol 2012; 86:11521-32.
19. Ding S, Veillette M, Coutu M, et al. A highly conserved residue of the HIV-1 gp120 inner domain is important for antibody-dependent cellular cytotoxicity responses mediated by anti-cluster A antibodies. J Virol 2016; 90:2127-34.
20. Durand M, Chartrand-Lefebvre C, Baril J G, et al. The Canadian HIV and Aging Cohort Study—determinants of increased risk of cardio-vascular diseases in HIV-infected individuals: rationale and study protocol. BMC Infect Dis 2017; 17:611.
21. Park J K, Kim J Y, Kwon H M, et al. Multidetector computed tomography for the evaluation of coronary artery disease; the diagnostic accuracy in calcified coronary arteries, comparing with IVUS imaging. Yonsei Med J 2014; 55:599-605.
22. Chen Z, Boldeanu I, Nepveu S, et al. In vivo coronary artery plaque assessment with computed tomography angiography: is there an impact of iterative reconstruction on plaque volume and attenuation metrics? Acta Radiol 2017; 58:660-9.
23. Boldeanu I, Sadouni M, Mansour S, et al. Prevalence and characterization of subclinical coronary atherosclerotic plaque with CT among individuals with HIV: results from the Canadian HIV and Aging Cohort study. Radiology 2021; 299:571-80.
24. Coutu M, Finzi A. HIV-1 gp120 dimers decrease the overall affinity of gp120 preparations for CD4-induced ligands. J Virol Methods 2015; 215-216:37-44.
25. Turcotte I, El-Far M, Sadouni M, et al. Association between the development of subclinical cardiovascular disease and human immunodeficiency virus (HIV) reservoir markers in people with HIV on suppressive antiretroviral therapy. Clin Infect Dis 2023; 76:1318-21.
26. Tolbert W D, Gohain N, Alsahafi N, et al. Targeting the late stage of HIV-1 entry for antibody-dependent cellular cytotoxicity: structural basis for env epitopes in the C11 region.

Structure 2017; 25:1719-31.e4.
27. Veillette M, Desormeaux A, Medjahed H, et al. Interaction with cellular CD4 exposes HIV-1 envelope epitopes targeted by antibody-dependent cell-mediated cytotoxicity. J Virol 2014; 88:2633-44.
28. Huang J, Kang B H, Ishida E, et al. Identification of a CD4-binding-site antibody to HIV that evolved near-pan neutralization breadth. Immunity 2016; 45:1108-21.
29. Tolbert W D, Gohain N, Veillette M, et al. Paring down HIV env: design and crystal structure of a stabilized inner domain of HIV-1 gp120 displaying a major ADCC target of the A32 region. Structure 2016; 24:697-709.
30. Sherburn R, Tolbert W D, Gottumukkala S, et al. Incorporating the cluster A and V1V2 targets into a minimal structural unit of the HIV-1 envelope to elicit a cross-clade response with potent Fc-effector functions. Vaccines (Basel) 2021; 9:975.
31. Wu X, Yang Z Y, Li Y, et al. Rational design of envelope identifies broadly neutralizing human monoclonal antibodies to HIV-1. Science 2010; 329:856-61.
32. Falutz J. Frailty in people living with HIV. Curr HIV/AIDS Rep 2020; 17:226-36.
33. Grund B, Baker J V, Deeks S G, et al. Relevance of interleukin-6 and D-dimer for serious non-AIDS morbidity and death among HIV-positive adults on suppressive antiretroviral therapy. PLoS One 2016; 11:e0155100.
34. Veillette M, Coutu M, Richard J, et al. The HIV-1 gp120 CD4-bound conformation is preferentially targeted by antibody-dependent cellular cytotoxicity-mediating antibodies in sera from HIV-1-infected individuals. J Virol 2015; 89:545-51.
35. Decker J M, Bibollet-Ruche F, Wei X, et al. Antigenic conservation and immunogenicity of the HIV coreceptor binding site. J Exp Med 2005; 201:1407-19.
36. Castilho J L, Shepherd B E, Koethe J, et al. CD4+/CD8+ ratio, Age, and risk of serious noncommunicable diseases in HIV-infected adults on antiretroviral therapy. AIDS 2016; 30:899-90.
37. Fernandez Soto J, Romero-Jimenez M J, Alarcón Garcia J C, Bonet Estruch E, Sánchez Ramos J L, Castaño López M. Predictors of subclinical atherosclerosis in HIV. BMC Infect Dis 2023; 23:17.
38. Lataillade M, Lalezari J P, Kozal M, et al. Safety and efficacy of the HIV-1 attachment inhibitor prodrug fostemsavir in heavily treatment-experienced individuals: week 96 results of the phase 3 BRIGHTE study. Lancet HIV 2020; 7:e740-e51.
39. Kozal M, Aberg J, Pialoux G, et al. Fostemsavir in adults with multidrug-resistant HIV-1 infection. N Engl J Med 2020; 382:1232-43.
40. Cohn L B, Chomont N, Deeks S G. The biology of the HIV-1 latent reservoir and implications for cure strategies. Cell Host Microbe 2020; 27:519-30.
41. Imamichi H, Smith M, Adelsberger J W, et al. Defective HIV-1 proviruses produce viral proteins. Proc Natl Acad Sci USA 2020; 117:3704-10.
42. Pollack R A, Jones R B, Pertea M, et al. Defective HIV-1 proviruses are expressed and can be recognized by cytotoxic T lymphocytes, which shape the proviral landscape. Cell Host Microbe 2017; 21:494-506.e4.
43. Kuniholm J, Armstrong E, Bernabe B, et al. Intragenic proviral elements support transcription of defective HIV-1 proviruses. PLoS Pathog 2021; 17:e1009982.
44. Busman-Sahay K, Starke C E, Nekorchuk M D, Estes J D. Eliminating HIV reservoirs for a cure: the issue is in the tissue. Curr Opin HIV AIDS 2021; 16:200-8.
45. McLaughlin M M, Ma Y, Scherzer R, et al. Association of viral persistence and atherosclerosis in adults with treated HIV infection. JAMA Netw Open 2020; 3:e2018099.
46. Yang X, Mahony E, Holm G H, Kassa A, Sodroski J. Role of the gp120 inner domain beta-sandwich in the interaction between the human immunodeficiency virus envelope glycoprotein subunits. Virology 2003; 313:117-25.
47. Wu X, Yang Z Y, Li Y, et al. Rational design of envelope identifies broadly neutralizing human monoclonal antibodies to HIV-1. Science 2010; 329:856-61.
48. Vandergeeten C, Fromentin R, Merlini E, et al. Cross-clade ultrasensitive PCR-based assays to measure HIV persistence in large-cohort studies. J Virol 2014; 88:12385-96.
49. Fantuzzi L, Canini I; Belardelli F, Gessani S. HIV-1 gp120 Stimulates the Production of β-Chemokines in Human Peripheral Blood Monocytes Through a CD4-Independent Mechanism. J Immunol (2001) 166 (9): 5381-5387.

1. A method for detecting the presence of soluble gp120 (sgp120) in a sample comprising:
(a) contacting the sample with a first sgp120-binding molecule that specifically binds to an epitope within the N-terminal 7-stranded or 8-stranded β-sandwich structure of gp120, thereby forming a first complex between the first sgp120-binding molecule and sgp120 if sgp120 is present in the sample;
(b) contacting the sample of (a) with a detection agent comprising:
(i) a first detection agent comprising a second sgp120-binding molecule that specifically binds to an epitope within the constant region 1 and 2 (C1-C2) portion of the cluster A region of gp120, conjugated to a third sgp120-binding molecule that specifically binds to a CD4-binding domain of gp120;
(ii) a second detection agent comprising a fourth sgp120-binding molecule that specifically binds to an epitope within the bridging sheet of the Co-Receptor Binding Site (CoRBS) of gp120, conjugated to the third sgp120-binding molecule;
(iii) a third detection agent comprising a fifth sgp120-binding molecule that specifically binds to an epitope formed by residues 97, 124, 198, 275-276, 278-283, 355, 365-368, 370-371, 425-427, 430, 455-463, 469 and 471-476 of gp120; or
(iv) a combination of (i) to (iii); and
(c) detecting the presence of a second complex between the detection agent and the first complex, wherein the presence of the second complex is indicative that sgp120 is present in the sample.

2. The method of claim 1, wherein the first sgp120-binding molecule is antibody clone C11 or an antibody or an antigen-binding fragment thereof that competes with antibody clone C11.

3. (canceled)

4. The method of claim 2, wherein the first sgp120-binding molecule is antibody clone C11, L9-i2, DH677.3 or N12-i3.

5. The method of claim 1, wherein the second sgp120-binding molecule is antibody clone A32 or an antibody or an antigen-binding fragment thereof that competes with antibody clone A32.

6. (canceled)

7. The method of claim 5, wherein the second sgp120-binding molecule is antibody clone A32, L9-i1 or N5-i5.

8. The method of claim 1, wherein the fourth sgp120-binding molecule is antibody clone 17b or an antibody or an antigen-binding fragment thereof that competes with antibody clone 17b.

9. (canceled)

10. The method of claim 8, wherein the fourth sgp120-binding molecule is antibody clone 17b, X5, L9-i3, N5-i1, N5-i3, N5-i4, N5-i8, N10-i1.1, N10-i5.3, N12-i1, N12-i2, N12-i4, N12-i5, N12-i7, N12-i8.

11. The method of claim 1, wherein the fifth sgp120-binding molecule is antibody clone N6 or an antibody or an antigen-binding fragment thereof that competes with antibody clone N6.

12. (canceled)

13. The method of claim 11, wherein the fifth sgp120-binding molecule is antibody clone N6, N49P6, N49P7 or N49P11.

14. The method of claim 1, wherein the third sgp120-binding molecule is a polypeptide comprising domains 1 and 2 of human CD4 receptor.

15-17. (canceled)

18. The method of claim 1, wherein the first sgp120-binding molecule is attached to a solid support.

19. The method of claim 1, wherein the first, second and/or third detection agent is conjugated to a detectable label.

20-22. (canceled)

23. The method of claim 1, wherein the sample is a biological sample.

24. (canceled)

25. A kit comprising:
(a) a first soluble gp120 (sgp120)-binding molecule that specifically binds to an epitope within the N-terminal 8-stranded β-sandwich structure, of gp120;
(b) a detection agent comprising:
(i) a first detection agent comprising a second sgp120-binding molecule that specifically binds to an epitope within the constant region 1 and 2 (C1-C2) portion of the cluster A region of gp120, conjugated to a third sgp120-binding molecule that specifically binds to a CD4-binding domain of gp120;
(ii) a second detection agent comprising a fourth sgp120-binding molecule that specifically binds to an epitope within the bridging sheet of the Co-Receptor Binding Site (CoRBS) of gp120, conjugated to the third sgp120-binding molecule;
(iii) a third detection agent comprising a fifth sgp120-binding molecule that specifically binds to an epitope formed by residues 97, 124, 198, 275-276, 278-283, 355, 365-368, 370-371, 425-427, 430, 455-463, 469 and 471-476 of gp120; or
(iv) a combination of (i) to (iii).

26. The kit of claim 25, wherein the first sgp120-binding molecule is antibody clone C11 or an antibody or an antigen-binding fragment thereof that competes with antibody clone C11; wherein the second sgp120-binding molecule is antibody clone A32 or an antibody or an antigen-binding fragment thereof that competes with antibody clone A32; wherein the fourth sgp120-binding molecule is antibody clone 17b or an antibody or an antigen-binding fragment thereof that competes with antibody clone 17b; and/or wherein the fifth sgp120-binding molecule is antibody clone N6 or an antibody or an antigen-binding fragment thereof that competes with antibody clone N6.

27. (canceled)

28. The kit of claim 26, wherein the first sgp120-binding molecule is antibody clone C11, L9-i2 or N12-i3; the second sgp120-binding molecule is antibody clone A32, L9-i1 or N5-i5; the fourth sgp120-binding molecule is antibody clone 17b, X5, L9-i3, N5-i1, N5-i3, N5-i4, N5-i8, N10-i1.1, N10-i5.3, N12-i1, N12-i2, N12-i4, N12-i5, N12-i7, N12-i8, N12-i10, N12-i17, N12-i18, or N12-i9; and/or the fifth sgp120-binding molecule is antibody clone N6, N49P6, N49P7 or N49P1.

29-37. (canceled)

38. The kit of claim 25, wherein the third sgp120-binding molecule is a polypeptide comprising domains 1 and 2 of human CD4 receptor.

48. The kit of claim 25, which further comprises instructions for detecting the presence of sgp120 in a sample according to a method comprising:
(a) contacting the sample with the first sgp120-binding molecule, thereby forming a first complex between the first sgp120-binding molecule and sgp120 if sgp120 is present in the sample;
(b) contacting the sample of (a) with a detection agent comprising:
(i) the first detection agent;
(ii) the second detection agent;
(iii) the third detection agent; or
(iv) a combination of (i) to (iii); and
(c) detecting the presence of a second complex between the detection agent and the first complex, wherein the presence of the second complex is indicative that sgp120 is present in the sample.

49. (canceled)

50. A method of treating HIV-associated inflammaging or chronic immune activation in an HIV-infected individual, the method comprising administering an effective amount of an HIV-1 attachment inhibitor to the HIV-infected individual suffering from HIV-associated inflammaging or chronic immune activation identified by a method comprising: performing the method defined in claim 1 on a biological sample from the HIV-infected individual, wherein the presence of sgp120 in the sample is indicative that the individual suffers from or is at risk of suffering from HIV-associated inflammaging or chronic immune activation.

51. The method of claim 50, wherein the HIV-1 attachment inhibitor is temsavir or fostemsavir.

52-55. (canceled)


SEQUENCE LISTING (44 sequences)

SEQ ID NO: 1 - protein, 463 aa, Homo sapiens
EVQLVESGGGLVKPGGSLRLSCAASGFTFSSYSMNWVRQAPGRGLEWVSSISNTSTYIYYADSVEGRFTLSRDNAKNSLYLQMNSLRAEDTAVYYCARANQHFDWLLSLLGGYHYYGMDVWGQGTTVTVSSASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, Homo sapiens
DIVMTQSPLSLPVTPGEPASISCRSSQSLLHSNGYNYLDWYLQKPGQSPQLLIYLGSNRASGVPDRFIGSGSGTDFTLKISRVEAEDVGVFYCMQALQAVGFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 448 aa, Homo sapiens
QVQLVQSGAEVKKPGSSVRVSCKASGGSFSRYTVNWVRQAPGQGLEWMARFIPIFNMPDYAPKFQGRITITADESTSTAYLELSSLTSDDTAVYYCASRQHHEYFQEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 215 aa, Homo sapiens
EIVLTQSPGTLSMSPGERATLSCRASRTVSSSNLAWYQQKPGQAPRLLIYDVSSRATGIPDRFSGRGSGTDFTLTISRLEPEDFAVYYCQQYGTSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 678 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASNTGGGGSGGGGSGGGGSGGGGTGGGGSGGGGSGGGGTGGGGSQVQLQESGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 210 aa, Homo sapiens
VLTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAP

SEQ ID NO: 7 - protein, 468 aa, Homo sapiens
MGWSCIILFLVATATGVHSQVQLQQWGAGLLKPSETLSLTCGVYGESLSGHYWSWVRQPPGKRLEWIGEIKHNGSPNYHPSLKSRVTISLDMSKNQFSLNLTSVTAADTAVYFCARRSNWPYLPFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 232 aa, Homo sapiens
MGWSCIILFLVATATGVHCVIWMTQSPSFVSASVGDRVTITCRASQGISSYLAWYQQKPGKAPKLVIYAASTLQSGVPSRFSGSGSGTEFTLTISSLQPEDFATYYCQHLIGLRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, Homo sapiens
EVQLVESGGGLVQPGGSLRLSCAASGFTFSTYAMSWVRQAPGKGLEWVSSINNSGRNTFSADSVKGRFTISRDNSKNTLFLVMNSLRAEDTAVYYCAKDLRLGGGSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, Homo sapiens
QSALTQPASVSGSPGQSITISCTGTSSDVGSYNFVSWYQQHPGKAPKLMIYEVSERPSGISNRFSGSKSGNTASLTISGLQAEDEADYYCSSYAGSTTFRVFGGGTKLTVRGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 683 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASNTGGGGSGGGGSGGGGSGGGGTGGGGSGGGGSGGGGTGGGGSEVQLVQSGAEVKKPGSSVKVSCKASGDTFIRYSFTWVRQAPGQGLEWMGRIITILDVAHYAPHLQGRVTITADKSTSTVYLELRNLRSDDTAVYFCAGVYEGEADEGEYDNNGFLKHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, Homo sapiens
ELELTQSPATLSVSPGERATLSCRASESVSSDLAWYQQKPGQAPRLLIYGASTRATGVPARFSGSGSGAEFTLTISSLQSEDFAVYYCQQYNNWPPRYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 13 - protein, 231 aa, Homo sapiens
EVQLVQSGAEVKKPGSSVKVSCKASGGTFSNYAINWVRQAPGQGLEWMGGIIPIFNIAHYAQRFQGRVSITADESTSTAYMELSSLRSEDTAVFYCASPYPNDYNDYAPEEGMSWYFDLWGRGTLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 14 - protein, 214 aa, Homo sapiens
DIQMTQSPSTLSASVGDRVTITCRASQSISNWLAWYQQKPGRAPKLLMYKASSLKSGVPSRFSGSGSGTEFTLTISSLQSDDFATYYCQQHDSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 15 - protein, 219 aa, Homo sapiens
EVQLVQSGAEVKKPGATVKISCKASGYTFSDFYMYWVRQAPGKGLEWMGLIDPEDADTMYAEKFRGRVTITADTSTDTGYLELSSLRSEDTAVYYCAADPWELNAFNVWGQGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 16 - protein, 212 aa, Homo sapiens
DIQMTQSPSSVSASVGDRVTITCRASQDISTWLAWYQQKPGKAPKLLIYAASTLQSGVPSRFSGSGSGTDFSLTINSLQPEDFATYYCQQANSFFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGE

SEQ ID NO: 17 - protein, 235 aa, Homo sapiens
EVQLVQSGAEVNKPGSSVKVSCQASGATLNSHAFSWVRQAPGQGLEWMAGIIPIFGSSHYAQKFRGRVTISADESTRTVYLHLRGLRSDDTAVYYCASNSIAGVAAAGDYADYDGGYYYDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 18 - protein, 213 aa, Homo sapiens
QSILTQPPSVSAAPGQKVTISCSGSSSNIGNNDVSWYQQFPGTVPKLVIYENNERPSGIPDRFSGSKSGTSATLGITGLQTGDEADYYCGTWDSSLSAVVFGGGSKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPT

SEQ ID NO: 19 - protein, 462 aa, Homo sapiens
QVQLVQSGAEVKKPGSSVKVSCRASRGTFSSYGITWVRQAPGQGLEWMGGIIPIFDVTNYAQNFQGRVAITTDAEMSTAYMELRSLKSEDSAVYYCASDSRDFSYYEPGTSYSHYYNIMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 215 aa, Homo sapiens
EIVMMQSPGTLSLSPGERATLSCRASQSVSSSYLAWYQQKPGQAPRLLIYGASSRATGIPDRFSGSGSGTDFTLTISRLEPEDFAVYYCQQYGSSPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 235 aa, Homo sapiens
EVQLLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 215 aa, Homo sapiens
ELVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKLEIKRTVAAPSVFIFPPSDEQLKSGTASVVCLLNNFYPREAKVQWKVDNALQSGNSQESVTEQDSRDSTYSLGSTLTLSKADYEKHKVYACEVTHQGLSSPVTKSFNRGEC

SEQ ID NO: 23 - protein, 225 aa, Homo sapiens
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 24 - protein, 210 aa, Homo sapiens
YIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 169 aa, Homo sapiens
AGLMQSGAVMKNSGASVRVSCQADGYDFIDYVIHWFRQRRGEGLEWLGWMNPSGGGTNYPRPFQGKVTMTRDTSTETAYLDVRGLTYDDTAVYYCVRDRANGSGRRRFESVNWFLDLWGRGTQITVVSASTKGPSVFPLAPSSKSTSGGTAALGCLVKDYFPEPVTVSW

SEQ ID NO: 26 - protein, 205 aa, Homo sapiens
QSALTQPRSVSASPGQSVTISCTGTHNYVSWCQQKPGQAPKLLIYDFNKRPSGVSDRFSGSTSGNTASLTISGLQADDEGHYFCWAFENIGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - protein, 476 aa, Homo sapiens
MGWSCIILFLVATATGVHSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 205 aa, Homo sapiens
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 120 aa, Homo sapiens
QIELVQSGTEVKRPGASVRISCASSGYRFTNYFIHWVRQAPGRGLEWMGWMNPLHGGVNYSGRFQGRVTMTRDIYTETSFMVLSGLTSDDTAIYFCTRGRDGYDDGFHPWGQGTLVTVSA

SEQ ID NO: 30 - protein, 100 aa, Homo sapiens
YVQSQSPSTLYASVGDKITITCRSSQSGWKAWYQQKPGKAPKLLIYKSSILETGVPSRFSGSDSGTEFTLTISSLQPDDFATYYCQHFETFGQGTRVQVR

SEQ ID NO: 31 - protein, 120 aa, Homo sapiens; identical to SEQ ID NO: 29

SEQ ID NO: 32 - protein, 101 aa, Homo sapiens
CFVQSQSPSTLYASVGDKITITCRSSQSGWKAWYQQKPGKAPKLLIYKSSILETGVPSRFIGSDSGTEFTLTISSLQPDDFATYYCQHFETFGQGTRVQVR

SEQ ID NO: 33 - protein, 121 aa, Homo sapiens
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCARGPSGENYPFHYWGQGVRVVVSSP

SEQ ID NO: 34 - protein, 101 aa, Homo sapiens
ASALTQPASMSASPGQSVTISCSGTRHIISAWFQQYPGKPPKLIIFDDDKRPSGVPSRFSASRPGDTASLTISNVQPEDEATYICNTYEFFGGGTRLTVLS

SEQ ID NO: 35 - protein, 221 aa, Homo sapiens
HVQLVQSGGGVKKIGAAVRISCEVTGYKFMDQLINWVRQAPGQGLEWMGWMNPTYGQVNYSWRFEGRVTMTRDMDTETAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 36 - protein, 201 aa, Homo sapiens
QPASMSASPGQSVTISCSGTRHIISAWFQQYPGKPPKLIIFDDDKRPSGVPSRFSASRPGDTASLTISNVQPEDEATYICNTYEFFGGGTRLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 450 aa, Homo sapiens
HVQLVQSGGGVKKIGAAVRISCEVSGYNFMDQFINWVRQAPGQGLEWMGWMNPIYGQVNYSWRFQGRVTMTRDMYTDTAFMELRGLRVDDTAVYYCARGPSGENYPFHYWGQGVRVVVSSPSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 203 aa, Homo sapiens
LTQPASMSASPGQSVTISCSGTRHIISAWFQQYPGKPPKLIIFDDDKRPSGVPSRFSASRPGDTASLTISNVQPEDEATYICNTYEFFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 39 - protein, 129 aa, Homo sapiens
SAELVQSGAVVKKPGTSVKVSCQAYGYTFTDYLIHWLRQAPGQGLEWMGWMNPVYGQVNYAQNFQGRVSMTRDIYRETAFLEVRDLKTDDTGTYYCVRDTGDGSRRHFDSINWFLDLWGRGTWIRVAPA

SEQ ID NO: 40 - protein, 100 aa, Homo sapiens
QCVLTQPRSVSGSPGQSVTISCTGTHNYVSWCQHHPGNAPKLLLYDFDKRPSGISDRFSGSRSGNTASLTISGLQPEDEADYFCWAFEAFGGGTKVLVLR

SEQ ID NO: 41 - protein, 228 aa, Homo sapiens
QVQLVQSGAEVQKPGASVKVSCKASGYTFASYDINWVRQATGQGLEWMGWMNPKTGNTGYAQKFQGRVTLTRNTSISTAYMELTSLRSEDTAVYYCATYRIIAAVGYRYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 42 - protein, 214 aa, Homo sapiens
DIQLTQSPSSLSASVGDSVTITCRASQGFGNYLAWYQQRPGKVPEVLIYAATTLQSGVPSRFSGSGSGTDFTLTISSLQPEDVATYYCQKYNSAPFTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 511 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 44 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI